SEQUENCE LISTING (41 sequences)

SEQ ID NO: 1 - protein, 5 aa, Artificial Sequence
  VH-CDR1
SYGVH

SEQ ID NO: 2 - protein, 16 aa, Artificial Sequence
  VH-CDR2
VIWSGGDTDYNAAFIS

SEQ ID NO: 3 - protein, 15 aa, Artificial Sequence
  VH-CDR3
NRGDIYYDFTYAMDY

SEQ ID NO: 4 - protein, 11 aa, Artificial Sequence
  VL-CDR1
RASGNIQNYLA

SEQ ID NO: 5 - protein, 7 aa, Artificial Sequence
  VL-CDR2
SAKTLAD

SEQ ID NO: 6 - protein, 9 aa, Artificial Sequence
  VL-CDR1
QHFWSTPYT

SEQ ID NO: 7 - protein, 123 aa, Artificial Sequence
  VH
QVQLKQSGPGLVQPSQSLSITCTVSGFSLTSYGVHWVRQSPGKGLEWLGVIWSGGDTDYNAAFISRLSISKDNSKSQLFFKMNSLRATDTAIYYCARNRGDIYYDFTYAMDYWGQGTSVTVSS

SEQ ID NO: 8 - protein, 108 aa, Artificial Sequence
  VL
DIQMTQSPASLSASVGETVTITCRASGNIQNYLAWYQQTQGKSPQLLVYSAKTLADGVPSRFSGSGSGTQYSLKINSLQPEDFGSYYCQHFWSTPYTFGGGTKLEIKR

SEQ ID NO: 9 - protein, 294 aa, Pseudomonas aeruginosa
  pcrV
MEVRNLNAARELFLDELLAASAAPASAEQEELLALLRSERIVLAHAGQPLSEAQVLKALAWLLAANPSAPPGQGLEVLREVLQARRQPGAQWDLREFLVSAYFSLHGRLDEDVIGVYKDVLQTQDGKRKALLDELKALTAELKVYSVIQSQINAALSAKQGIRIDAGGIDLVDPTLYGYAVGDPRWKDSPEYALLSNLDTFSGKLSIKDFLSGSPKQSGELKGLSDEYPFEKDNNPVGNFATTVSDRSRPLNDKVNEKTTLLNDTSSRYNSAVEALNRFIQKYDSVLRDILSAI

SEQ ID NO: 10 - protein, 585 aa, Escherichia coli
  Flagelline
MAQVINTNSLSLITQNNINKNQSALSSSIERLSSGLRINSAKDDAAGQAIANRFTSNIKGLTQAARNANDGISVAQTTEGALSEINNNLQRIRELTVQATTGTNSDSDLDSIQDEIKSRLDEIDRVSGQTQFNGVNVLAKDGSMKIQVGANDGETITIDLKKIDSDTLGLNGFNVNGKGTITNKAATVSDLTSAGAKLNTTTGLYDLKTENTLLTTDAAFDKLGNGDKVTVGGVDYTYNAKSGDFTTTKSTAGTGVDAAAQAADSASKRDALAATLHADVGKSVNGSYTTKDGTVSFETDSAGNITIGGSQAYVDDAGNLTTNNAGSAAKADMKALLKAASEGSDGASLTFNGTEYTIAKATPATTTPVAPLIPGGITYQATVSKDVVLSETKAAAATSSITFNSGVLSKTIGFTAGESSDAAKSYVDDKGGITNVADYTVSYSVNKDNGSVTVAGYASATDTNKDYAPAIGTAVNVNSAGKITTETTSAGSATTNPLAALDDAISSIDKFRSSLGAIQNRLDSAVTNLNNTTTNLSEAQSRIQDADYATEVSNMSKAQIIQQAGNSVLAKANQVPQQVLSLLQG

SEQ ID NO: 11 - protein, 495 aa, Salmonella enterica subsp. enterica serovar Typhimurium [sequence omitted]

SEQ ID NO: 12 - protein, 494 aa, Salmonella enterica subsp. enterica serovar Typhimurium
  Flagelline
AQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 13 - protein, 4 aa, Artificial Sequence
  Linker
GAAG

SEQ ID NO: 14 - protein, 273 aa, Artificial Sequence
  FLAMOD
MKAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNGAAGATTTENPLQKIDAALAQVDTLRSDLGAVQNRFNSAITNLGNTVNNLTSARSRIEDSDYATEVSNMSRAQILQQAGTSVLAQANQVPQSVLSLLR

SEQ ID NO: 15 - protein, 8 aa, Artificial Sequence
  Conservative residues
QRVRELAV

SEQ ID NO: 16 - protein, 5 aa, Artificial Sequence
  VH-CDR1
SYAMN

SEQ ID NO: 17 - protein, 17 aa, Artificial Sequence
  VH-CDR2
AITISGITAYYTDSVKG

SEQ ID NO: 18 - protein, 15 aa, Artificial Sequence
  VH-CDR3
EEFLPGTHYYYGMDV

SEQ ID NO: 19 - protein, 11 aa, Artificial Sequence
  VL-CDR1
RASQGIRNDLG

SEQ ID NO: 20 - protein, 7 aa, Artificial Sequence
  VL-CDR2
SASTLQS

SEQ ID NO: 21 - protein, 9 aa, Artificial Sequence
  VL-CDR3
LQDYNYPWT

SEQ ID NO: 22 - protein, 8 aa, Artificial Sequence
  VH-CDR1
GFTFSSYA

SEQ ID NO: 23 - protein, 8 aa, Artificial Sequence
  VH-CDR2
IRGSGYSS

SEQ ID NO: 24 - protein, 16 aa, Artificial Sequence
  VH-CDR3
AKERSVTAYYYYGMDV

SEQ ID NO: 25 - protein, 6 aa, Artificial Sequence
  VL-CDR1
QDISSF

SEQ ID NO: 26 - protein, 9 aa, Artificial Sequence
  VL-CDR3
QQLKSYPLT

SEQ ID NO: 27 - protein, 8 aa, Artificial Sequence
  VH-CDR1
GFTFNTYA

SEQ ID NO: 28 - protein, 8 aa, Artificial Sequence
  VH-CDR2
IGGSGYST

SEQ ID NO: 29 - protein, 15 aa, Artificial Sequence
  VH-CDR3
AKEGNIVALYWYFDL

SEQ ID NO: 30 - protein, 6 aa, Artificial Sequence
  VL-CDR1
QSISRY

SEQ ID NO: 31 - protein, 9 aa, Artificial Sequence
  VL-CDR3
QQSSTTPLT

SEQ ID NO: 32 - protein, 8 aa, Artificial Sequence
  VH-CDR1
GFTFSDHE

SEQ ID NO: 33 - protein, 8 aa, Artificial Sequence
  VH-CDR2
IGSGVVTM

SEQ ID NO: 34 - protein, 15 aa, Artificial Sequence
  VH-CDR3
ARDRGYYFGSEAFHY

SEQ ID NO: 35 - protein, 6 aa, Artificial Sequence
  VL-CDR1
QSISNW

SEQ ID NO: 36 - protein, 9 aa, Artificial Sequence
  VL-CDR3
QQYKSYSLT

SEQ ID NO: 37 - protein, 8 aa, Artificial Sequence
  VH-CDR1
GFTFSTFA

SEQ ID NO: 38 - protein, 8 aa, Artificial Sequence
  VH-CDR2
IGASGYST

SEQ ID NO: 39 - protein, 16 aa, Artificial Sequence
  VH-CDR3
AKEYSVSSNYYYGMDV

SEQ ID NO: 40 - protein, 6 aa, Artificial Sequence
  VL-CDR1
QTIRRY

SEQ ID NO: 41 - protein, 9 aa, Artificial Sequence
  VL-CDR3
QQTYSIPIT

(19) United States
(12) Patent Application Publication
(10) Pub. No.: US 2023/0372416 A1
(43) Pub. Date: Nov. 23, 2023

(54) COMBINATION OF INHALED ANTIBODIES AND IMMUNOMODULATORY AGENTS FOR THE TREATMENT OR PREVENTION OF RESPIRATORY INFECTIONS

(71) Applicants: UNIVERSITÉ DE TOURS, Tours Cedex 1 (FR); INSTITUT NATIONAL DE RECHERCHE POUR L'AGRICULTURE, L'ALIMENTATION ET L'ENVIRONNEMENT, Paris (FR); INSERM (INSTITUT NATIONAL DE LA SANTÉ ET DE LA RECHERCHE MÉDICALE), Paris (FR)

(72) Inventors: Nathalie HEUZE-VOURC'H, Tours (FR); Thomas SECHER, Luynes (FR); Muriel THOMAS, Igny (FR)

(73) Assignees: UNIVERSITÉ DE TOURS, Tours Cedex 1 (FR); INSTITUT NATIONAL DE RECHERCHE POUR L'AGRICULTURE, L'ALIMENTATION ET L'ENVIRONNEMENT, Paris (FR); INSERM (INSTITUT NATIONAL DE LA SANTÉ ET DE LA RECHERCHE MÉDICALE), Paris (FR)

(21) Appl. No.: 18/248,120

(22) PCT Filed: Oct. 8, 2021
(86) PCT No.: PCT/FR2021/051758

§ 371 Date: Apr. 6, 2023

(30) Foreign Application Priority Data
Oct. 8, 2020 (FR) FR2010296

(51) Int. Cl. A61K 35/747; A61P 31/04; A61K 39/40; A61P 11/00; A61K 9/00

(52) U.S. Cl. CPC A61K 35/747; A61P 31/04; A61K 39/40; A61P 11/00; A61K 9/0073; A61K 2039/544

(57) ABSTRACT

A combination of agents capable of binding an infectious agent, such as an antibody, an antibody derivative, or an antibody mimetic, and immunomodulatory agents, such as probiotic strains, for the treatment or prevention of respiratory infections, in particular bacterial respiratory infections. The agents capable of binding an infectious agent are administered by inhalation.

### TECHNICAL FIELD

The present invention relates to the treatment and prevention of respiratory infections in a subject. In particular, the present invention relates to the treatment or prevention of respiratory infections caused by bacteria, for example, antibiotic resistant bacteria, and more particularly, Pseudomonas aeruginosa.

### STATE OF THE ART

Respiratory diseases are a major public health issue worldwide, with 4 families of respiratory diseases ranking among the top 10 causes of death and accounting for nearly 9 million deaths per year (i.e., ⅙ of total deaths), in addition to significant morbidity and a high economic impact (€380 billion annually in Europe). These diseases include in particular chronic obstructive pulmonary disease (COPD), lung cancer, acute respiratory infections and tuberculosis.

In particular, respiratory infections account for a quarter of deaths from respiratory diseases in Europe. Globally, respiratory infections (excluding tuberculosis) caused 2.4 million deaths in 2016, positioning them as the 6th most deadly disease in humans (all ages combined), and as the leading cause of death in children under 5 years of age. The prevention and treatment of respiratory infections therefore represent a major public health issue.

Although antibiotics have revolutionized the management of these diseases, their efficacy is continuously decreasing due to the rapid emergence of resistance phenomena. Given the low number of new antibiotics marketed, government agencies are calling for efforts to develop new anti-infectious therapeutic strategies.

Thus, the Inventors have developed an innovative strategy based on a combination of antibodies administered by inhalation and immunomodulatory agents to fight respiratory infections. In particular, the Inventors have shown that the pulmonary administration of an antibody directed against an infectious agent, in combination with probiotic strains, makes it possible to treat the respiratory infection caused by the infectious agent and to prevent its recurrence.

### SUMMARY OF THE INVENTION

An object of the present invention is a combination of at least one agent capable of binding an infectious agent and at least one immunomodulatory agent, for use in treating or preventing a respiratory infection in a subject, wherein the at least one agent capable of binding the infectious agent is for administration by inhalation to the subject.

According to one embodiment, the at least one agent capable of binding the infectious agent is for administration by inhalation to the subject. According to one embodiment, the at least one agent capable of binding the infectious agent is in a form suitable for administration by inhalation to the subject.

According to one embodiment, the at least one agent capable of binding the infectious agent is selected from the group comprising or consisting of an antibody, an antibody derivative and an antibody mimetic, preferably the at least one agent capable of binding the infectious agent is an antibody or an antibody derivative.

According to one embodiment, the at least one immunomodulatory agent is selected from the group comprising or consisting of a probiotic strain, a mixture of probiotic strains, a Toll-like receptor agonist, a NOD-like receptor agonist, a RIG-like receptor agonist, a cytokine or a mixture of cytokines, a chemokine or a mixture of chemokines, an adjuvant such as chitosan, a flagellin, a flagellin variant, a polypeptide comprising or consisting of one or more flagellin fragment(s), a CpG oligodeoxynucleotide (CpG ODN), α-galactosylceramide (α-Gal-Cer), aluminum salts (aluminum hydroxide, aluminum phosphate, and potassium aluminum sulfate), MF59, AS03, polyinosinic-polycytidylic acid, a polyphosphazene, an antibody directed against immune checkpoints such as CTLA-4, PD-1, PD-L1 or CD137 and mixtures thereof.

According to one embodiment, the at least one immunomodulatory agent is or comprises a probiotic strain or a mixture of probiotic strains selected from the Lactobacillaceae family, preferably from the species Lactobacillus murinus.

According to one embodiment, the at least one immunomodulatory agent is or comprises a probiotic strain selected from the strains deposited on Apr. 14, 2015 at the Collection Nationale de Cultures de Microorganismes (CNCM) under the numbers CNCM I-4967 and CNCM I-4968, or the strain deposited at the CNCM on Apr. 16, 2018 under number CNCM I-5314, or a mixture thereof.

According to one embodiment, the at least one immunomodulatory agent is or comprises a mixture of the probiotic strains CNCM I-4967 and CNCM I-4968. According to one embodiment, the at least one immunomodulatory agent is or comprises a mixture of probiotic strains CNCM I-4967 and CNCM I-5314. According to one embodiment, the at least one immunomodulatory agent is or comprises a mixture of probiotic strains CNCM I-5314 and CNCM I-4968. According to one embodiment, the at least one immunomodulatory agent is or comprises a mixture of probiotic strains CNCM I-4967, CNCM I-4968 and CNCM I-5314.

According to one embodiment, the infectious agent is selected from the group comprising or consisting of viruses, bacteria, fungi and parasites, preferably the infectious agent is at least temporarily extracellular.

According to one embodiment, the infectious agent is a bacterium, preferably a bacterium resistant to one or more antibiotics, more preferably a bacterium selected from the group ESKAPE, even more preferably a bacterium selected from the group comprising or consisting of Pseudomonas aeruginosa, Klebsiella pneumoniae, Staphylococcus aureus and Acinetobacter baumanii, even more preferably Pseudomonas aeruginosa.

According to one embodiment, the at least one agent capable of binding the infectious agent is directed against a molecule present on the surface of a bacterium. According to one embodiment, the at least one agent capable of binding the infectious agent is directed against a molecule present on the surface of Pseudomonas aeruginosa, preferably the at least one agent capable of binding the infectious agent is directed against a protein of the type III secretion system of Pseudomonas aeruginosa.

According to one embodiment, said respiratory infection is an acute respiratory infection, preferably an acute lower respiratory tract infection, more preferably bronchitis, bronchiolitis, pneumonia (including nosocomial pneumopathy, community-acquired pneumopathy, or ventilator-assisted pneumopathy), influenza, or pertussis.

According to one embodiment, the subject suffers from a chronic respiratory pathology, preferably a chronic respiratory pathology selected from the group comprising or consisting of chronic obstructive pulmonary disease (COPD), pulmonary interstitial diseases, lung cancer, asthma (adult and pediatric), bronchiectasis, rare and orphan lung diseases such as cystic fibrosis, and pulmonary vascular diseases.

The invention also relates to a composition comprising or consisting essentially of a combination of at least one agent capable of binding an infectious agent and at least one immunomodulatory agent for use as described above.

The invention also relates to a kit of parts comprising at least two parts, the first part comprising at least one agent capable of binding the infectious agent and the second part comprising at least one immunomodulatory agent for use in the treatment or prevention of a respiratory infection in a subject, wherein the at least one agent capable of binding the infectious agent is to be administered by inhalation to the subject.

According to one embodiment, the at least one agent capable of binding the infectious agent is selected from the group comprising or consisting of an antibody, an antibody derivative and an antibody mimetic, preferably the at least one agent capable of binding the infectious agent is an antibody or an antibody derivative.

According to one embodiment, the at least one immunomodulatory agent is selected from the group comprising or consisting of a probiotic strain, a mixture of probiotic strains, a Toll-like receptor agonist, a NOD-like receptor agonist, a RIG-like receptor agonist, a cytokine or a mixture of cytokines, a chemokine or a mixture of chemokines, an adjuvant such as chitosan, a flagellin a polypeptide comprising or consisting of one or more flagellin fragment(s), a CpG oligodeoxynucleotide (CpG ODN), α-galactosylceramide (α-Gal-Cer), aluminum salts (aluminum hydroxide, aluminum phosphate, and potassium aluminum sulfate), MF59, AS03, polyinosinic-polycytidylic acid, a polyphosphazene, an antibody directed against immune checkpoints such as CTLA-4, PD-1, PD-L1, or CD137 and mixtures thereof.

### DEFINITIONS

In the present invention, the terms below are defined as follows:
“Adnectin”: refers to an artificial antigen-binding protein based on the 10th extracellular domain of fibronectin type III.
“Affibody”: refers to an affinity protein based on a 58 amino acid protein domain derived from an immunoglobulin G binding domain of protein A from the bacterium Staphylococcus aureus.
“Affilin”: refers to an artificial antigen-binding protein with a structure derived from human gamma-B-crystallin.
“Affitin”: refers to an antigen-binding protein, with a structure derived from the DNA-binding protein Sac7d from Sulfolobus acidocaldarius. Examples of affitins include, but are not limited to, Nanofitins developed by Affilogic. According to one embodiment, the at least one agent capable of binding an infectious agent is an affitin, for example, a Nanofitin.
“Atrimer”: refers to an artificial antigen-binding protein based on the structure of a trivalent human molecule, tetranectin.
“Immunomodulatory agent”: refers to a substance characterized by its ability to directly modulate the immune system, either by stimulating it (the immunomodulatory agent may then be referred to as an “immunostimulatory agent”) or curbing it. According to one embodiment, the substance is added to assist or cooperate with the immune system to enhance immune responses against the infectious agent. According to one embodiment, the immunomodulatory agent may therefore be referred to as an “immunostimulating agent”.
“Infectious agent”: refers to an agent that causes an infectious disease. Infectious agents include, in particular, viruses, bacteria, fungi and parasites. As used in this invention, the term “infectious agent” includes emerging infectious agents (or pathogens).
“Agonist”: refers to a molecule that binds to and activates a receptor to induce a biological response.
“Antibiotics”: refers to a natural or synthetic substance that destroys or inhibits the growth of bacteria. As used in the present invention, an antibiotic agent is not considered an as immunomodulatory agent. According to one embodiment, an antibiotic agent is not considered an immunostimulatory agent.
“Anticalin”: refers to an antibody mimetic for which the binding specificity is derived from lipocalins. Anticalins can also be produced as proteins that bind to 2 targets, in which case they are called duocalins.
“Antibody” (which may also be referred to as “Immunoglobulin”): refers to a molecule that binds specifically to an antigen. An antibody is generally made up of two light chains (L) and two heavy chains (H) linked together by covalent or non-covalent bonds. The generic term “Immunoglobulin” (Ig) includes 5 distinct classes of antibodies that can be distinguished biochemically. Within the meaning of the present invention, all 5 classes of antibodies are covered by the invention; the following discussion will focus primarily on the immunoglobulin G class Immunoglobulin G comprises two light polypeptide chains with a molecular weight of about 23,000 Daltons (Da), and two heavy polypeptide chains with a molecular weight of about 53,000-70,000 Da. The 4 chains are connected by disulfide bridges in a Y-shaped configuration. The light chains of an antibody are classified as kappa ([κ]) or lambda ([λ]). Each class of heavy chain can bind to either a [κ] or [λ] light chain. In general, the heavy and light chains are covalently linked to each other, and the tail regions of the 2 heavy chains are linked to each other by covalent disulfide bonds or noncovalent bonds when the immunoglobulins are produced by hybridomas, B cells, or genetically modified host cells. In the heavy chain, the amino acid sequence extends from the N-terminal part at the branches of the Y to the C-terminal part at the end of the chain. Heavy chains are classified as gamma, mu, alpha, delta, or epsilon (γ, μ, α, δ, ϑ respectively) with subclasses (e.g., γ1-γ4) among them. The nature of this heavy chain determines the class (or isotype) of the antibody, namely IgG, IgM, IgA IgD, or IgE, respectively. Modified versions of each of these classes and isotypes are within the scope of the skilled person and are therefore included in the present invention. The variable portion of an antibody allows the antibody to selectively recognize antigens and to specifically bind the epitope to the antigens. Indeed, the light chain variable domain and the heavy chain variable domain combined form a variable region that defines a 3-dimensional antigen binding domain. This domain is present at the end of each branch of the Y. More specifically, the antigen-binding domain is defined by 3 regions that determine complementarity (complementarity determining regions, CDR) present on each of the heavy and light chains.
“Single domain antibody”: refers to the smallest functional antigen-binding unit of an antibody, which corresponds to the variable regions of the heavy or light chains of the antibody.
“Bispecific antibody” and “Multispecific antibody”: refer to a type of antibody modified to bind to two (bispecific) or more (multispecific) distinct antigens.
“Chimeric antibody”: refers to an antibody in which both types of chains (heavy and light) are chimeric as a result of engineering modifications. A chimeric chain is a chain containing a foreign variable region, i.e., from a species other than human or synthetic, linked to a constant region of human origin.
“Humanized antibody”: refers to an antibody in which both types of chains are humanized as a result of engineering modifications. A humanized chain is one in which the complementarity determining regions (CDRs) of the variable regions are foreign, i.e., non-human or synthetic, while the rest of the chain is of human origin.
“Antibody of human origin”: refers to an antibody in which both chains are of human origin.
“Avimer”: refers to an artificial antigen-binding protein derived from membrane receptor domains.
“Bacteria”: refers to a prokaryotic organism, i.e., a single-celled organism without a nucleus, whose genome consists of DNA. This consists of a single chromosome, which may be accompanied by plasmids (circular DNA). There are two general categories of bacteria based on the biochemical characteristics of their cell wall: Gram-positive and Gram-negative bacteria.
“Fungus”: refers to a heterotrophic eukaryotic organism providing nutrition by absorption and producing spores. There are two main groups: ascomycetes and basidiomycetes.
“Chitosan”: refers to a linear polysaccharide composed of the random distribution of 13-(1-4)-linked D-glucosamine and N-acetyl-D-glucosamine.
“DARPin” (Designed Ankyrin Repeat Proteins): refers to a genetically produced antibody mimetic derived from an ankyrin protein.
“Antibody derivative”: refers to a molecule derived from an antibody as defined above, and includes bispecific and multispecific antibodies, antibody fragments, single domain antibodies, unibodies and nanobodies.
“Diabody”: refers to a dimer of antibody fragments, each polypeptide consisting of a variable region of a heavy chain and a variable region of a light chain. Triabodies and tetrabodies are trimers and tetramers of fragments respectively.
“About”: preceding a numeral or number, means plus or minus 10% of the value of said numeral or number.
“Pharmaceutically acceptable excipient”: refers to a substance that does not produce an adverse, allergic or undesirable reaction when administered to a subject. This includes all solvents, dispersion media, coatings, antibacterial and antifungal agents, isotonic agents, delayed absorption agents and other similar substances. For human administration, preparations must meet the criteria of sterility, pyrogenicity, and general safety and purity standards required by regulatory agencies such as the FDA or EMA.
“Flagellin”: refers to a protein contained in a variety of Gram-positive or Gram-negative bacterial species. Sources of flagellin include, but are not limited to, Escherichia, (e.g., E. coli), Enterobacter, Erwinia, Klebsiella, Proteus, Salmonella (e.g., Salmonella enterica serovar Typhimurium), Serratia (e.g, Serratia marcescans), Shigella, Bacilli (e.g., B. subtilis and B. licheniformis), Pseudomonas (e.g., P. aeruginosa), and Streptomyces. Amino acid sequences and nucleotide sequences of flagellins are publicly available in the NCBI Genbank database, for example under the following accession numbers: AAL20871, NP_310689, BAB58984, AAO85383, AAA27090, NP_461698, AAK58560, YP_001217666, YP_002151351, YP_001250079, AAA99807, CAL35450, AAN74969, and BAC44986. Flagellin sequences from these and other species are included in the term “flagellin” used here. Thus, sequence differences between species are included in this term.
“Antibody fragment”: refers to a portion or region of an antibody comprising fewer amino acids than the intact antibody and which may bind to the same antigen and/or may compete with the antibody from which it is derived for binding to the antigen. The antibody fragments included in a non-limiting manner in the present invention are Fab, Fab′, F(ab′)₂, Fd, variable fragments (Fv), single chain variable fragments (scFv), diabodies, triabodies and tetrabodies.
“Fab Fragment”: refers to an antibody fragment formed by the entire light chain (light chain variable domain (VL) and light chain constant domain (CL)) and a portion of the heavy chain (heavy chain variable domain (VH) and heavy chain constant domain 1 (CH1)).
“Fab′ fragment”: refers to an antibody fragment formed by the reduction of an F(ab′)₂ fragment. This fragment contains reactive sulfhydryl groups.
“F(ab′)₂ fragment”: refers to the combination of two Fab fragments linked by a small part of the constant parts of the heavy chains, the hinge region.
“Fc fragment”: refers to an antibody fragment formed by the constant domains 2 and 3 of the heavy chains (CH2 and CH3) beyond the hinge region.
“Fd fragment”: refers to an antibody fragment formed by the heavy chain variable region (VH) and the first heavy chain constant domain (CH1).
“Variable Fragment (Fv)”: refers to an antibody fragment formed solely by the variable regions of the heavy and light chains (VL and VH).
“Single-chain variable fragment (scFv)”: refers to antibody fragments comprising the variable regions of the heavy and light chains (VH and VL) connected in a single amino acid chain. In one embodiment, an scFv fragment comprises a linker peptide between the VH and VL variable regions allowing the fragment to have the proper structure to bind antigen (Plüickthun, 1994. Antibodies from Escherichia coli. In Rosenberg & Moore (Eds.), The pharmacology of monoclonal antibodies. Handbook of Experimental Pharmacology, 113:269-315. Springer: Berlin, Heidelberg).
“Fynomeric”: refers to an artificial antigen-binding protein derived from the SH3 domain of the Fyn protein.
“Identity” or “identical”: when used in a relationship between the sequences of two or more polypeptides, means the degree of sequence relatedness between the polypeptides, as determined by the number of matches between chains of two or more amino acid residues. Identity measures the percentage of identical matches between the smallest sequences of two or more sequences with gap alignments (if any) processed by a particular mathematical model or computer program (i.e., “algorithms”) The identity of related polypeptides can be readily calculated by methods known to the skilled person. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al, SIAM J. Applied Math. 48, 1073 (1988). The preferred methods for determining identity are designed to give the greatest correspondence between the sequences being tested. The methods of identity determination are described in publicly available computer programs. Preferred computer program methods for determining the identity between two sequences include the GCG package, including GAP (Devereux et al. Acid. Res. \2, 387 (1984); Genetics Computer Group, University of Wisconsin, Madison, Wis.), BLASTP, BLASTN, and FASTA (Altschul et al., J. MoI. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm can also be used to determine identity.
“Respiratory infection”: refers to an infection affecting any of the component structures of the respiratory system, i.e., the nose, throat, larynx, trachea, bronchi, or lungs by an infectious agent.
“Inhalation”: refers to the administration of a substance into the respiratory tract, including in particular an aerosol, spray, squirt, drip or any other form suitable for administration directly into the respiratory tract.
“Antibody mimetic”: refers to artificially created molecules that can specifically bind to antigens, as antibodies, but do not share the structures of antibodies. According to one embodiment, the antibody mimetics are coupled to an antibody Fc fragment. Antibody mimetics of the present application include, but are not limited to, affibodies, affilins, affitins, adnectins, atrimers, DARPins, anticalins, avimers, fynomers and versabodies.
“Morbidity”: refers to the set of effects subsequent to a disease or trauma, often referred to as sequelae.
“Nanobody”: refers to an antibody-derived therapeutic protein containing the structural and functional properties of naturally occurring heavy chain antibodies. These heavy chain antibodies can contain a single variable domain and 2 constant domains (CH2 and CH3).
“Therapeutically effective amount”: refers to the necessary and sufficient amount of the combination of the present invention to be administered in a subject allowing for the prevention of respiratory infection, slowing or stopping the progression, worsening, deterioration of at least one of the symptoms of respiratory infection. This amount administered may result in relief of the symptoms of the respiratory infection, cure of the infection, or prevention of the respiratory infection.
“Parasite”: refers to an organism living on or in another organism, called a host, at the expense of the host.
“Peptide”: refers to a linear polymer of amino acids, usually composed of less than 50 amino acids linked together by peptide bonds. Amino acid polymers composed of more than 50 amino acids are generally referred to as polypeptides, while the term “Protein” generally refers to an assembly of peptides or polypeptides.
“Polypeptide comprising or consisting of one or more flagellin fragment(s)” or “Flagellin polypeptide”: refer to a polypeptide comprising or consisting of one or more flagellin fragment(s), said polypeptide retaining its ability to bind and activate Toll-like receptor 5 (TLR5). As used in this application, the term “Toll-like receptor 5” or “TLR5” refers to a TLR5 of any species, preferably a human TLR5. According to one embodiment, a flagellin polypeptide as described herein comprises the flagellin domains involved in TLR5 signaling. The term “flagellin domain” includes naturally occurring flagellin domains and functional conservative variants thereof. Flagellin domains involved in TLR5 signaling are well known to those skilled in the art, see for example Smith et al. (2003) Nat. Immunol. 4: 1247-1253 (e,g., amino acids 78-129, 135-173, and 394-444 of S. typhimurium flagellin or modified homologues or forms thereof).
“Reinfection” or “secondary infection” or “second infection”: mean a new infection of an organism by an infectious agent that caused a primary infection (primary infection being the first attack on an organism by an agent infectious agent that has been treated with the combination of the present invention) or by an infectious agent of the same species that caused the primary infection. Reinfections may be repeated.
“Probiotic strain”: refers to a live microorganism that, when administered in effective amounts by a host, confers a health benefit. Probiotics include yeast and bacteria.
“Subject”: refers to a mammal, preferably a human In one embodiment, the subject may be a “patient”, i.e., a warm-blooded animal, preferably a human, awaiting or receiving medical care, who has undergone a medical procedure, or who is being monitored for the development of a respiratory infection. In another embodiment, the subject is a warm-blooded animal, preferably a human, who does not have a respiratory infection.
“Treatment” or “treat” refers to both therapeutic treatment and prophylactic or preventive measures, the purpose of which is to inhibit or destroy the infectious agent responsible for a respiratory infection and/or to prevent the development of a respiratory infection or its recurrence. Subjects in need of treatment include subjects with a pre-existing respiratory infection, subjects at risk of developing a respiratory infection, and subjects in whom a respiratory infection must be prevented. According to one embodiment, a subject is considered successfully treated for a respiratory infection if, after receiving a therapeutically effective amount of the combination of the present invention, the subject observably or measurably shows a reduction in the number of infectious agents (responsible for the respiratory infection), a reduction in symptoms, a reduction in morbidity and/or an improvement in quality of life. According to one embodiment, the subject is considered treated if, after receiving a therapeutically effective amount of the combination of the present invention, said subject does not develop a respiratory infection as a result of contact with the infectious agent targeted by said combination or develops a respiratory infection that is less severe than an infection generally observed in the absence of treatment. The above assessment parameters are readily measurable by routine procedures familiar to a physician.
“Unibody”: refers to an antibody fragment derived from IgG4 antibodies, in which the hinge region has been removed. Removal of the hinge region results in a molecule that is half the size of a conventional IgG4 antibody, and has a monovalent binding domain in contrast to the bivalent binding domain of IgG4 antibodies.
“Flagellin variants”: refers to functional conservative variants, i.e., variants in which one or more given amino acid residue(s) has/have been modified without altering the overall conformation and function of the flagellin, which includes, but is not limited to, the replacement of one amino acid with another having similar properties (such as, e.g., polarity, hydrogen bonding potential, acidity, basicity, hydrophobicity, aromaticity, and the like).
“Versabody”: refers to an artificial antigen-binding protein. It is a small protein of 3 to 5 kDa with more than 15% cysteines, which forms a high density disulfide scaffold, replacing the typical hydrophobic core of proteins.
“Virus”: refers to an infectious agent that can replicate only by entering a living cell and using its cellular machinery. A virus is composed of a viral genome possibly enveloped in a protein shell or capsid, or even an envelope. The viral genome can be a single or double strand of DNA or RNA.

### DETAILED DESCRIPTION

The present invention relates to a combination of at least one agent capable of binding an infectious agent and at least one immunomodulatory agent for use in the treatment or prevention of a respiratory infection in a subject, wherein the at least one agent capable of binding the infectious agent is to be administered (or is formulated for administration) by inhalation to the subject.

In one embodiment, the at least one agent capable of binding the infectious agent is an antibody, an antibody derivative (e.g., an antibody fragment) or an antibody mimetic.

According to one embodiment, the combination of the present invention comprises one immunomodulatory agent. According to one embodiment, the combination of the present invention comprises a plurality of immunomodulatory agents.

According to one embodiment, the at least one immunomodulatory agent is a probiotic strain or a mixture of probiotic strains. According to one embodiment, the immunomodulatory agent is a mixture of 2, 3 or 4 probiotic strains, preferably a mixture of 2 probiotic strains.

According to one embodiment, the one or more probiotic strains are bacteria.

According to one embodiment, the probiotic strain(s) are living or active bacteria, i.e., capable of multiplying.

According to another embodiment, the probiotic strain(s) are inactive bacteria, i.e., not able to multiply.

According to the present invention, the probiotic strain(s) are said to be live or active if the bacteria are capable of multiplying under culture conditions suitable for the growth of said bacteria.

According to one embodiment, the probiotic strain(s) are bacteria selected from Gram-positive bacteria. According to one embodiment, the probiotic strain(s) are bacteria selected from Gram negative bacteria.

Examples of Gram-positive and Gram-negative bacteria that may be used in the present invention include, but are not limited to, the Lactobacillaceae family, the Enterococcaceae family, the Bifidobacteriaceae family and the Enterobacteriaceae family.

According to one embodiment, the at least one immunomodulatory agent is or comprises a probiotic strain or a mixture of probiotic strains selected from the Lactobacillaceae family.

According to one embodiment, the at least one immunomodulatory agent is or comprises a probiotic strain or a mixture of probiotic strains selected from the Bifidobacteriaceae family.

According to one embodiment, the at least one immunomodulatory agent is or comprises a probiotic strain or a mixture of probiotic strains selected from the Enterococcaceae family, preferably from the Enterococcus faecalis species.

According to one embodiment, the at least one immunomodulatory agent is or comprises a probiotic strain or a mixture of probiotic strains selected from the Enterobacteriaceae family, preferably from the species Escherichia coli.

According to one embodiment, the at least one immunomodulatory agent is or comprises a probiotic strain or a mixture of probiotic strains selected from the genus Lactobacillus.

According to one embodiment, the at least one immunomodulatory agent is or comprises a probiotic strain selected from the species Lactobacillus rhamnosus and/or a genomically related species.

According to one embodiment, the at least one immunomodulatory agent is or comprises a probiotic strain selected from the species Lactobacillus salivarius and/or a genomically related species.

According to one embodiment, the at least one immunomodulatory agent is or comprises a mixture of probiotic strains selected from Lactobacillus rhamnosus and Lactobacillus salivarius species.

According to one embodiment, the at least one immunomodulatory agent is a mixture of a probiotic strain selected from Lactobacillus rhamnosus species (and/or a species close in genomic terms) and a probiotic strain selected from the species Lactobacillus salivarius (and/or a species close in genomic terms).

According to one embodiment, a species close to Lactobacillus rhamnosus and Lactobacillus salivarius in genomic terms is a species whose 16S ribosomal RNA has at least 97% (e.g., about 98%, 99%, or more than 99%) sequence homology with the 16S ribosomal RNA of the Lactobacillus rhamnosus and Lactobacillus salivarius species, respectively.

According to one embodiment, a species close to Lactobacillus rhamnosus and Lactobacillus salivarius in genomic terms is a species whose total genome has at least 97% (e.g., about 98%, 99%, or more than 99%) sequence homology with the complete genome of the Lactobacillus rhamnosus and Lactobacillus salivarius species respectively.

According to one embodiment, the at least one immunomodulatory agent is or comprises a probiotic strain or mixture of probiotic strains selected from the species Lactobacillus murinus and/or a genomically related species.

Examples of probiotic strains of the species Lactobacillus murinus include, but are not limited to, strain 313^{T} and strain CNCM I-5314 deposited with the CNCM on Apr. 16, 2018 on behalf of Institut National de la Recherche Agronomique (now Institut National de Recherche pour l'Agriculture, l'Alimentation et l'Environnement as of Jan. 1, 2020).

Strain 313^{T} is described in Zheng et al (“A taxonomic note on the genus Lactobacillus: Description of 23 novel genera, emended description of the genus Lactobacillus Beijerinck 1901 and union of Lactobacillaceae and Leuconostocaceae,” Int J Syst Evol Microbiol, 2020, 70(4): 2782-2858). It is available at the ATCC (American Type Culture Collection) under reference 35020^{T}, at the CCUG (Culture Collection University of Gothenburg) under reference 33904^{T}, at the CIP (Collection de l'Institut Pasteur) under reference 104818^{T}, at the CNRZ (Centre national de recherches zootechniques) under reference 220^{T}, at the DSMZ (German Collection of Microorganisms and Cell Cultures) under reference 20452^{T}, at the NBRC (NITE Biological Resource Center) under reference 14221^{T}, in the JCM (Japan Collection of Microorganisms) under reference 1717^{T} and in the LMG (Belgian Coordinated Collections of Micro-organisms) under reference 14189^{T}.

Strain CNCM I-5314 is described in Bernard-Raichon L. et al (“A Pulmonary Lactobacillus murinus Strain Induces Th17 and RORγt+ Regulatory T Cells and Reduces Lung Inflammation in Tuberculosis”, 2021 Sep. 3:ji2001044).

In addition, sequence analyses by Illumina NGS sequencing showed that strains CNCM I-4967 and CNCM I-4968, deposited at CNCM on Apr. 14, 2015 on behalf of Institut National de la Recherche Agronomique (now Institut National de Recherche pour l'Agriculture, l'Alimentation et l'Environnement as of Jan. 1, 2020) also belong to the species Lactobacillus murinus.

According to one embodiment, a species close to Lactobacillus murinus in genomic terms is a species whose 16S ribosomal RNA has at least 97% (e.g., about 98%, 99%, or more than 99%) sequence homology with the 16S ribosomal RNA of the species Lactobacillus murinus.

According to one embodiment, a species close to Lactobacillus murinus in genomic terms is a species whose total genome has at least 97% (e.g., about 98%, 99%, or more than 99%) sequence homology with the complete genome of the species Lactobacillus murinus.

The names used above refer to the pre-2020 nomenclature of the Lactobacillaceae, as a new classification has been proposed recently (Zheng et al, “A taxonomic note on the genus Lactobacillus: Description of 23 novel genera, emended description of the genus Lactobacillus Beijerinck 1901 and union of Lactobacillaceae and Leuconostocaceae”, Int J Syst Evol Microbiol, 2020, 70(4): 2782-2858). The person skilled in the art is able to make the correspondence between an old and a new taxonomy. If the taxonomy were to be changed again, the person skilled in the art could adapt the taxonomy changes to derive the bacteria designated in the present invention.

For example, the names Lactobacillus rhamnosus and Lactobacillus salivarius used in the old nomenclature correspond to Lacticaseibacillus rhamnosus and Ligilactobacillus salivarius in the new nomenclature, respectively. In addition, the name Lactobacillus murinus used in the old nomenclature corresponds to Ligilactobacillus murinus of the new nomenclature.

According to one embodiment, the at least one immunomodulatory agent is or comprises the probiotic strain deposited at the CNCM on Apr. 14, 2015 under the number CNCM I-4967, in the name of Institut National de la Recherche Agronomique (now Institut National de Recherche pour l'Agriculture, l'Alimentation et l'Environnement as of Jan. 1, 2020).

According to one embodiment, the at least one immunomodulatory agent is or comprises the probiotic strain deposited at the CNCM on Apr. 14, 2015 under the number CNCM I-4968 in the name of Institut National de la Recherche Agronomique (now Institut National de Recherche pour l'Agriculture, l'Alimentation et l'Environnement as of Jan. 1, 2020).

According to one embodiment, the at least one immunomodulatory agent is or comprises the probiotic strain deposited at the CNCM on Apr. 16, 2018 under the number CNCM I-5314 in the name of Institut National de la Recherche Agronomique (now Institut National de Recherche pour l'Agriculture, l'Alimentation et l'Environnement as of Jan. 1, 2020).

According to a particular embodiment, the at least one immunomodulatory agent of the present invention is or comprises a mixture of the 2 probiotic strains deposited at CNCM on Apr. 14, 2015 under numbers CNCM I-4967 and CNCM I-4968. According to a particular embodiment, the at least one immunomodulatory agent is or comprises a mixture of the probiotic strain deposited with the CNCM on Apr. 14, 2015 under number CNCM I-4967 and the probiotic strain deposited with the CNCM on Apr. 16, 2018 under number CNCM I-5314. According to one embodiment, the at least one immunomodulatory agent is or comprises a mixture of the probiotic strain deposited at the CNCM on Apr. 14, 2015 under number CNCM I-4968 and the probiotic strain deposited at CNCM on Apr. 16, 2018 under number CNCM I-5314.

According to a particular embodiment, the at least one immunomodulatory agent of the present invention is or comprises a mixture of the 3 probiotic strains deposited at CNCM on Apr. 14, 2015 under numbers CNCM I-4967 and CNCM I-4968 and on Apr. 16, 2018 under number CNCM I-5314.

According to one embodiment, the at least one immunomodulatory agent is or comprises a chemical molecule, a peptide molecule, a protein molecule, a sugar, a lipid or a nucleic acid encoding an immunomodulatory agent.

According to one embodiment, the at least one immunomodulatory agent is or comprises a molecule that binds to Pattern-Recognition Receptors (PRRs).

According to one embodiment, the molecule binding to the pattern recognition receptors is selected from agonists of the Toll-like receptors (TLR); agonists of NOD-like receptors (NODs) and agonists of RIG-like receptors (RIGs).

Examples of agonists of these receptors that may be used in the present invention include, but are not limited to, lipopeptide diacyl and triacyl, peptidoglycan, lipoproteins, single and double stranded RNA, lipopolysaccharide (LPS), flagellin, non-methylated CpG DNA, polyinosinic-polycytidylic acid (poly I:C) and lipoteichoic acids. Another example of agonists for these receptors is a polypeptide comprising one or more flagellin fragment(s) (“flagellin polypeptide”), or a flagellin variant.

According to one embodiment, the at least one agent capable of binding the infectious agent is combined with one or more molecules that bind to the molecular pattern recognition receptors and one or more probiotic strains.

According to one embodiment, the at least one immunomodulatory agent is or comprises a molecule that binds to receptors on the surface of cells and modulates the immune response, such as cytokines, chemokines and the similar.

According to one embodiment, the at least one immunomodulatory agent is or comprises a cytokine or a mixture of cytokines. Examples of cytokines that may be used in the context of the present invention include, but are not limited to, interleukin (IL)-1b, IL-6, IL-7, IL-12, IL-15, IL-17, IL-23, Tumor Necrosis Factor α (TNF α), type I interferon (IFN type I).

According to one embodiment, the at least one agent capable of binding the infectious agent is combined with one or more cytokines and one or more probiotic strains.

According to one embodiment, the at least one immunomodulatory agent is or comprises a chemokine or a mixture of chemokines. Examples of chemokines that may be used in the context of the present invention include but are not limited to, CCLS (Chemokine C-C motif ligand 5), CCL27 (Chemokine C-C motif ligand 27), CXCL9 (Chemokine C-X-C motif ligand 9), CXCL10 (Chemokine C-X-C motif ligand 10) and CXCL11 (Chemokine C-X-C motif ligand 11), CXCL16 (Chemokine C-X-C motif ligand 10).

According to one embodiment, the at least one agent capable of binding the infectious agent is combined with one or more chemokines and one or more probiotic strains.

According to one embodiment, the at least one immunomodulatory agent is or comprises an adjuvant, for example chitosan.

According to one embodiment, the at least one agent capable of binding the infectious agent is combined with one or more adjuvants and one or more probiotic strains.

According to one embodiment, the at least one agent capable of binding the infectious agent is combined with chitosan and one or more probiotic strains.

Examples of immunomodulatory agents that may be used in the present invention include, but are not limited to, flagellin, an oligodeoxynucleotide CpG (CpG ODN), α-galactosylceramide (α-Gal-Cer), aluminum salts (aluminum hydroxide, aluminum phosphate, and potassium aluminum sulfate), MF59, AS03, polyinosinic-polycytidylic acid and a polyphosphazene. Another example of an immunomodulatory agent that may be used in the present invention is a polypeptide comprising or consisting of one or more flagellin fragment(s) (which may be referred to as “flagellin polypeptide”) or a flagellin variant.

According to one embodiment, the at least one agent capable of binding the infectious agent is combined with flagellin and one or more probiotic strains. According to one embodiment, the at least one agent capable of binding the infectious agent is combined with a polypeptide comprising or consisting of one or more flagellin fragment(s) (i.e., “flagellin polypeptide”) and one or more probiotic strains. According to one embodiment, the at least one agent capable of binding the infectious agent is combined with a flagellin variant and one or more probiotic strains.

Examples of flagellins, flagellin variants or flagellin polypeptides include, but are not limited to, the peptides described in patents U.S. Pat. Nos. 6,585,980; 6,130,082; 5,888,810; 5,618,533; and 4,886,748 and patent applications US2003/0044429, WO2008097016, and WO2009156405, which are incorporated by reference in the present application.

Examples of flagellins include, but are not limited to, peptides comprising or consisting of the sequence SEQ ID NO: 10 (E. coli 0157:H7 flagellin), the sequence SEQ ID NO: 11 (S. typhimurium flagellin) or the sequence SEQ ID NO: 12 (S. typhimurium flagellin).

SEQ ID NO: 10
MAQVINTNSLSLITQNNINKNQSALSSSIERLSSGLRINSAKDDAAGQA
IANRFTSNIKGLTQAARNANDGISVAQTTEGALSEINNNLQRIRELTVQ
ATTGTNSDSDLDSIQDEIKSRLDEIDRVSGQTQFNGVNVLAKDGSMKIQ
VGANDGETITIDLKKIDSDTLGLNGFNVNGKGTITNKAATVSDLTSAGA
KLNTTTGLYDLKTENTLLTTDAAFDKLGNGDKVTVGGVDYTYNAKSGDF
TTTKSTAGTGVDAAAQAADSASKRDALAATLHADVGKSVNGSYTTKDGT
VSFETDSAGNITIGGSQAYVDDAGNLTTNNAGSAAKADMKALLKAASEG
SDGASLTFNGTEYTIAKATPATTTPVAPLIPGGITYQATVSKDVVLSET
KAAAATSSITFNSGVLSKTIGFTAGESSDAAKSYVDDKGGITNVADYTV
SYSVNKDNGSVTVAGYASATDTNKDYAPAIGTAVNVNSAGKITTETTSA
GSATTNPLAALDDAISSIDKFRSSLGAIQNRLDSAVTNLNNTTTNLSEA
QSRIQDADYATEVSNMSKAQIIQQAGNSVLAKANQVPQQVLSLLQG
SEQ ID NO: 11
MAQVINTNSLSLLTQNNLNKSQSALGTAIERLSSGLRINSAKDDAAGQA
IANRFTANIKGLTQASRNANDGISIAQTTEGALNEINNNLQRVRELAVQ
SANSTNSQSDLDSIQAEITQRLNEIDRVSGQTQFNGVKVLAQDNTLTIQ
VGANDGETIDIDLKQINSQTLGLDTLNVQQKYKVSDTAATVTGYADTTI
ALDNSTFKASATGLGGTDQKIDGDLKFDDTTGKYYAKVTVTGGTGKDGY
YEVSVDKTNGEVTLAGGATSPLTGGLPATATEDVKNVQVANADLTEAKA
ALTAAGVTGTASVVKMSYTDNNGKTIDGGLAVKVGDDYYSATQNKDGSI
SINTTKYTADDGTSKTALNKLGGADGKTEVVSIGGKTYAASKAEGHNFK
AQPDLAEAAATTTENPLQKIDAALAQVDTLRSDLGAVQNRFNSAITNLG
NTVNNLTSARSRIEDSDYATEVSNMSRAQILQQAGTSVLAQANQVPQNV
LSLLR
SEQ ID NO: 12
AQVINTNSLSLLTQNNLNKSQSALGTAIERLSSGLRINSAKDDAAGQAI
ANRFTANIKGLTQASRNANDGISIAQTTEGALNEINNNLQRVRELAVQS
ANSTNSQSDLDSIQAEITQRLNEIDRVSGOTQFNGVKVLAQDNTLTIQV
GANDGETIDIDLKQINSQTLGLDTLNVQQKYKVSDTAATVTGYADTTIA
LDNSTFKASATGLGGTDQKIDGDLKFDDTTGKYYAKVTVTGGTGKDGYY
EVSVDKTNGEVTLAGGATSPLTGGLPATATEDVKNVQVANADLTEAKAA
LTAAGVTGTASVVKMSYTDNNGKTIDGGLAVKVGDDYYSATQNKDGSIS
INTTKYTADDGTSKTALNKLGGADGKTEVVSIGGKTYAASKAEGHNFKA
QPDLAEAAATTTENPLQKIDAALAQVDTLRSDLGAVQNRFNSAITNLGN
TVNNLTSARSRIEDSDYATEVSNMSRAQILQQAGTSVLAQANQVPQNVL
SLLR

Thus, according to one embodiment, flagellin is a peptide of sequence SEQ ID NO: 10. According to one embodiment, the flagellin is a peptide of sequence SEQ ID NO: 11. According to one embodiment, the flagellin is a peptide of sequence SEQ ID NO: 12. These flagellins are described in international application WO2016102536.

According to one embodiment, flagellin or a flagellin variant has at least 70, 75, 80, 85, 90, 95, 97, 98 or 99% identity with any of the sequences SEQ ID Nos: 10-12.

According to one embodiment, flagellin or a flagellin variant comprises amino acid residues 89-96 of SEQ ID NO: 12 (i.e., residues involved in TLR5 detection). Thus, according to one embodiment, the flagellin or a flagellin variant comprises the sequence SEQ ID NO: 15.

SEQ ID NO: 15 =
QRVRELAV

Examples of flagellin polypeptides include, but are not limited to, polypeptides described in WO2009156405 and WO2016102536, which are incorporated by reference in the application.

According to one embodiment, the flagellin polypeptide comprises:
(a) an N-terminal peptide having at least 90% identity to the amino acid sequence beginning with the amino acid residue located at position 1 of SEQ ID NO: 12 and ending with an amino acid residue selected from the group consisting of any of the amino acid residues located at positions 99 to 173 of SEQ ID NO: 12 and
b) a C-terminal peptide having at least 90% identity to the amino acid sequence beginning with an amino acid residue selected from the group consisting of any of the amino acid residues located at positions 401 to 406 of SEQ ID NO : 12 and ending with the amino acid residue located at position 494 of SEQ ID NO: 12, wherein: said N-terminal peptide is directly linked to said C-terminal peptide, or said N-terminal peptide and said C-terminal peptide are indirectly linked to each other, with a spacer chain.

According to one embodiment, said N-terminal peptide is selected from the group consisting of or comprising amino acid sequences 1-99, 1-137, 1-160 and 1-173 of SEQ ID NO: 12.

According to one embodiment, said C-terminal peptide is selected from the group comprising or consisting of amino acid sequences 401-494 and 406-494 of SEQ ID NO: 12.

According to one embodiment, said N-terminal and C-terminal peptides comprise or consist of amino acid sequences 1-173 and 401-494 of SEQ ID NO: 12, respectively.

According to one embodiment, said N-terminal and C-terminal peptides comprise or consist of amino acid sequences 1-160 and 406-494 of SEQ ID NO: 12, respectively.

According to one embodiment, said N-terminal and C-terminal peptides comprise or consist of amino acid sequences 1-137 and 406-494 of SEQ ID NO: 12, respectively.

According to one embodiment, said N-terminal peptide and said C-terminal peptide are indirectly linked to each other with a spacer chain. A non-limiting example of a spacer chain is a sequence comprising or consisting of the peptide sequence SEQ ID NO: 13.

SEQ ID NO: 13 =
NH2-Gly-Ala-Ala-Gly-COOH

According to one embodiment, the asparagine amino acid residue located at position 488 of SEQ ID NO: 12 is replaced by a serine.

According to one embodiment, the flagellin polypeptide as described above comprises an additional methionine residue at the N-terminus.

According to one embodiment, the flagellin polypeptide as described above comprises an additional methionine (M) residue and an additional lysine (L) residue at the N-terminus.

According to one embodiment, the flagellin polypeptide comprises N-terminal and C-terminal peptides consisting of sequences 1-173 and 401-494 of SEQ ID NO: 12, said peptides being indirectly linked to each other with a spacer chain consisting of the peptide sequence SEQ ID NO: 13, said polypeptide comprising an additional methionine residue and an additional lysine residue at the N-terminal. According to one embodiment, the flagellin polypeptide is of sequence SEQ ID NO: 14 (FLAMOD).

SEQ ID NO: 14
MKAQVINTNSLSLLTQNNLNKSQSALGTAIERLSSGLRINSAKDDAAGQ
AIANRFTANIKGLTQASRNANDGISIAQTTEGALNEINNNLQRVRELAV
QSANSTNSQSDLDSIQAEITQRLNEIDRVSGQTQFNGVKVLAQDNTLTI
QVGANDGETIDIDLKQINSQTLGLDTLNGAAGATTTENPLQKIDAALAQ
VDTLRSDLGAVQNRFNSAITNLGNTVNNLTSARSRIEDSDYATEVSNMS
RAQILQQAGTSVLAQANQVPQSVLSLLR

According to one embodiment, the at least one agent capable of binding the infectious agent is combined with a CpG oligodeoxynucleotide and one or more probiotic strains.

According to one embodiment, the at least one agent capable of binding the infectious agent is combined with α-galactosylceramide and one or more probiotic strains.

According to one embodiment, the at least one agent capable of binding the infectious agent is combined with aluminum salt(s) and one or more probiotic strains.

According to one embodiment, the at least one agent capable of binding the infectious agent is combined with MF59 and one or more probiotic strains.

According to one embodiment, the at least one agent capable of binding the infectious agent is combined with AS03 and one or more probiotic strains.

According to one embodiment, the at least one agent capable of binding the infectious agent is combined with polyinosinic-polycytidylic acid and one or more probiotic strains.

According to one embodiment, the at least one agent capable of binding the infectious agent is combined with a polyphosphazene and one or more probiotic strains.

According to one embodiment, the at least one immunomodulatory agent is or comprises an antibody, preferably an antibody directed against immune system checkpoints such as CTLA-4, PD-1, PD-L1 or CD137.

According to one embodiment, the at least one agent capable of binding the infectious agent is combined with an antibody directed against immune system checkpoints such as CTLA-4, PD-1, PD-L1 or CD137 and one or more probiotic strains.

According to one embodiment, the combination of the present invention comprises one agent capable of binding the infectious agent.

According to one embodiment, the combination of the present invention comprises multiple agents capable of binding one or more infectious agent(s), for example two agents capable of binding one or two infectious agent(s).

According to one embodiment, the at least one agent capable of binding the infectious agent is directed against the infectious agent causing the respiratory infection.

According to one embodiment, the infectious agent is selected from the group comprising or consisting of viruses, bacteria, fungi and parasites.

Non-limiting examples of viruses responsible for respiratory infections include influenza, rhinovirus, parainfluenza, respiratory syncitial virus (RSV) and coronavirus. Examples of coronaviruses include, but are not limited to, Severe Acute Respiratory Syndrome Coronavirus (SARS-CoV) and Severe Acute Respiratory Syndrome Coronavirus 2 (SARS-CoV-2).

According to one embodiment, the infectious agent is a virus, preferably an influenza virus, RSV, SARS-CoV or SARS-CoV-2. According to one embodiment, the virus is temporarily extracellular during infection.

Non-limiting examples of bacteria responsible for respiratory infections include Streptococcus pneumoniae, Haemophilus influenzae, Moraxella catarrhalis, Pseudomonas aeruginosa, Klebsiella pneumoniae, Staphylococcus aureus and Acinetobacter baumanii.

Non-limiting examples of fungi causing respiratory infections include Pneumocystis jirovici, Cryptococcus neoformens, Aspergillus species and Histoplasma capsulatum capsulatum.

According to one embodiment, the infectious agent is at least temporarily extracellular, i.e., present or detectable outside the cell at least temporarily during infection.

According to one embodiment, the infectious agent is a bacterium. According to one embodiment, the infectious agent is an extracellular bacterium, i.e., during the infection, the bacterium is present or detectable outside the cells.

According to one embodiment, the infectious agent is a Gram-positive bacterium. According to another embodiment, the infectious agent of the present invention is a Gram negative bacterium.

According to one embodiment, the infectious agent is a bacterium resistant to one or more antibiotics.

A non-exhaustive list of antibiotics includes, penicillins (such as amoxicillin, clavulanic acid, ampicillin or cloxacillin); cyclins (such as doxycycline, minocycline or tetracycline); cephalosporins (such as cefadroxil, cefixime, cefpodoxime, ceftriazone or cefuroxime); carbapenems (such as doripenem, ertapenem, imipenem, or meropenem); aminoglycosides (such as gentamicin, amikacin, netilmicin, tobramycin, isepamicin, neomycin, streptomycin, or spectinomycin); macrolides (such as azithromycin, clarithromycin, roxithromycin or spiramycin); fluoroquinolones (such as ciprofloxacin, levofloxacin, moxifloxacin, norfloxacin or ofloxacin); or fosfomycin.

According to one embodiment, the infectious agent is a bacterium selected from the ESKAPE group.

According to the present invention, the ESKAPE group includes 6 multidrug-resistant pathogenic bacteria: Enterococcus faecium, Staphylococcus aureus, Klebsiella pneumoniae, Acinetobacter baumannii, Pseudomonas aeruginosa and Enterobacter spp.

According to one embodiment, the infectious agent is a bacterium selected from the group consisting of or including Pseudomonas aeruginosa, Klebsiella pneumoniae, Staphylococcus aureus and Acinetobacter baumanii.

According to one embodiment, the infectious agent is Pseudomonas aeruginosa,

According to one embodiment, the infectious agent is Pseudomonas aeruginosa of serotype O6, O11, O10, O2, or O1.

According to one embodiment, the infectious agent is strain PA103.

According to one embodiment, the at least one agent capable of binding the infectious agent used in the present invention is directed against a molecule present on the surface of the infectious agent, in particular of the infectious bacterium.

According to one embodiment, the at least one agent capable of binding the infectious agent is directed against a molecule expressed on the surface of Pseudomonas aeruginosa.

According to one embodiment, said molecule is a protein of a virulence system.

According to one embodiment, said molecule is a membrane receptor.

According to one embodiment, said molecule is a protein of a secretion system.

According to one embodiment, said molecule is a protein of a type III secretion system.

According to one embodiment, said molecule is perV.

According to one embodiment, said molecule is expressed by one or more strains of Pseudomonas aeruginosa. According to one embodiment, said molecule is expressed by strain PA103.

According to one embodiment, the at least one agent capable of binding the infectious agent used in the present invention is a molecule selected from the group comprising or consisting of an antibody, antibody derivative or antibody mimetic.

According to one embodiment, the at least one agent capable of binding the infectious agent is an antibody.

According to one embodiment, the at least one agent capable of binding the infectious agent is an antibody derivative.

According to one embodiment, an antibody derivative is an antibody fragment capable of binding an antigen.

Examples of antibody fragments include, but are not limited to, a variable fragment (Fv), a single chain variable fragment (scFv), a Fab fragment, a Fab′ fragment, an F(ab)′₂ fragment, an Fd fragment, a diabody, a triabody and a tetrabody.

According to one embodiment, the antibody derivative is a multispecific antibody, for example a bispecific antibody.

According to one embodiment, the antibody derivative is a single domain antibody, a unibody or a nanobody.

According to one embodiment, the at least one agent capable of binding the infectious agent is an antibody mimetic.

According to one embodiment, the antibody mimetic is coupled with an antibody Fc fragment.

Examples of antibody mimetics include, but are not limited to, affibodies, affilins, affitins, adnectins, atrimers, DARPins, anticalins, avimers, fynomers and versabodies.

According to one embodiment, the at least one agent capable of binding the infectious agent is an affitin.

According to one embodiment, the at least one antibody (or antibody derivative) used in the present invention is a chimeric, humanized or human-derived antibody (or derivative thereof).

According to one embodiment, the at least one antibody (or antibody derivative) used in the present invention belongs to one of the following isotypes: IgG, IgA, IgM, IgE or IgD, preferably IgG, IgA or IgM.

According to one embodiment, the at least one antibody (or antibody derivative) used in the present invention is a monoclonal antibody (or a derivative thereof). According to another embodiment, the at least one antibody (or antibody derivative) of the present invention is a polyclonal antibody (or derivative thereof).

According to one embodiment, the at least one antibody (or antibody derivative) of the present invention is directed against perV (SEQ ID NO: 9), i.e., an anti-PcrV antibody (or antibody derivative).

SEQ ID NO: 9
MEVRNLNAARELFLDELLAASAAPASAEQEELLALLRSERIVLAHAGQP
LSEAQVLKALAWLLAANPSAPPGQGLEVLREVLQQARRQPGAQWDLREF
LVSAYFSLHGRLDEDVIGVYKDVLQTQDGKRKALLDELKALTAELKVYS
VIQSQINAALSAKQGIRIDAGGIDLVDPTLYGYAVGDPRWKDSPEYALL
SNLDTFSGKLSIKDFLSGSPKQSGELKGLSDEYPFEKDNNVPGNFATTV
SDRSRPLNDKVNEKTTLLNDTTSSRYNSAVEALNRFIQKYDSVLRDILS
AI

Examples of antibodies directed against perV are described in WO2010091189, WO2013070615, WO2015196011, WO02064161, WO2020252029 (the contents of each of these applications are incorporated by reference).

Application WO2013070615 describes an anti-PcrV antibody comprising a heavy chain (VH) variable domain comprising the following 3 complementarity determining regions (CDR):

VH-CDR1:
(SEQ ID NO: 16)
SYAMN,
VH-CDR2:
(SEQ ID NO: 17)
AITISGITAYYTDSVKG,
VH-CDR3:
(SEQ ID NO: 18)
EEFLPGTHYYGMDV,
and
a variable light chain (VL) domain comprising
the following 3 CDRs:
VL-CDR1:
(SEQ ID NO: 19)
RASQGIRNDLG,
VL-CDR2:
(SEQ ID NO: 20)
SASTLQS,
VL-CDR3:
(SEQ ID NO: 21)
LQDYNYPWT.

For example, application WO2020252029 describes an anti-PcrV antibody comprising a heavy chain (VH) variable domain comprising the following 3 CDRs:

VH-CDR1:
(SEQ ID NO: 22)
GFTFSSYA,
VH-CDR2:
(SEQ ID NO: 23)
IRGSGYSS,
VH-CDR3:
(SEQ ID NO: 24)
AKERSVTAYYYGMDV,

and a variable light chain (VL) domain comprising the following 3 CDRs:

VL-CDR1:
(SEQ ID NO: 25)
QDISSE,
VL-CDR2:
TAS,
and
VL-CDR3:
(SEQ ID NO: 26)
QQLKSYPLT.

For example, application WO2020252029 describes an anti-PcrV antibody comprising a heavy chain (VH) variable domain comprising the following 3 CDRs:

VH-CDR1:
(SEQ ID NO: 27)
GFTFNTYA,
VH-CDR2:
(SEQ ID NO: 28)
IGGSGYST,
VH-CDR3:
(SEQ ID NO: 29)
AKEGNIVALYWYFDL,

and a variable light chain (VL) domain comprising the following 3 CDRs:

VL-CDR1:
(SEQ ID NO: 30)
QSISRY,
VL-CDR2:
AAS,
and
VL-CDR3:
(SEQ ID NO: 31)
QQSSTTPLT.

For example, application WO2020252029 describes an anti-PcrV antibody comprising a heavy chain (VH) variable domain comprising the following 3 CDRs:

VH-CDR1:
(SEQ ID NO: 32)
GFTFSDHE,
VH-CDR2:
(SEQ ID NO: 33)
IGSGVVTM,
VH-CDR3:
(SEQ ID NO: 34)
ARDRGYYFGSEAFHY,

and a variable light chain (VL) domain comprising the following 3 CDRs:

VL-CDR1:
(SEQ ID NO: 35)
QSISNW,
VL-CDR2:
KSS,
and
VL-CDR3:
(SEQ ID NO: 36)
QQYKSYSLT.

For example, application WO2020252029 describes an anti-PcrV antibody comprising a heavy chain (VH) variable domain comprising the following 3 CDRs:

VH-CDR1:
(SEQ ID NO: 37)
GFTFSTFA,
VH-CDR2:
(SEQ ID NO: 38)
IGASGYST,
VH-CDR3:
(SEQ ID NO: 39)
AKEYSVSSNYYYGMDV,

and a variable light chain (VL) domain comprising the following 3 CDRs:

VL-CDR 1:
(SEQ ID NO: 40)
QTIRRY,
VL-CDR 2:
AAS,
and
VL-CDR 3:
(SEQ ID NO: 41)
QQTYSIPIT.

According to one embodiment, the at least one antibody (or antibody derivative) used in the present invention is an antibody having CDRs as described above.

According to one embodiment, the at least one antibody (or antibody derivative) used in the present invention is mAb166 antibody (or a derivative thereof). The derivation and characterization of the mAb166 antibody is described in the prior art (Franck et al., Generation and characterization of a protective monoclonal antibody to Pseudomonas aeruginosa perV, The Journal of Infectious disease, July 2002; 186(1):64-73). The mAb166 antibody is commercially available (CreativeBiolabs, Reference MRO-156MZ; LGC Standards, Reference PTA-9180 hybridoma).

According to one embodiment, the mAb166 antibody comprises a heavy chain (VH) variable domain comprising the following 3 complementarity determining regions (CDRs):

VH-CDR 1:
(SEQ ID NO: 1)
SYGVH,
VH-CDR2:
(SEQ ID NO: 2)
VIWSGGDTDYNAAFIS,
VH-CDR3:
(SEQ ID NO: 3)
NRGDIYYDFTYAMDY,

and a variable light chain (VL) domain comprising the following 3 CDRs:

VL-CDR1:
(SEQ ID NO: 4)
RASGNIQNYLA,
VL-CDR2:
(SEQ ID NO: 5)
SAKTLAD,
VL-CDR3:
(SEQ ID NO: 6)
QHFWSTPYT.

According to one embodiment, the variable domains of the heavy and light chains of the mAb166 antibody are of sequences SEQ ID NO: 7 and SEQ ID NO: 8, respectively.

SEQ ID NO: 7
QVQLKQSGPGLVQPSQSLSITCTVSGFSLTSYGVHWRQSPGKGLEWLGV
IWSGGDTDYNAAFISRLSISKDNSQLFFKMNSLRATDTAIYYCARNRGD
IYYDFTYAMDYWGQGTSVTVSS
SEQ ID NO: 8
DIQMTOSPASLSASVGETVTITCRASGNIQNYLAWYQQTQGKSPQLLVY
SAKTLADGVPSRFSGSGTQYSLKINSLQPEDFGSYYCQHFWSTPYTFGG
GTKLEIKR

According to one embodiment, the at least one antibody (or antibody derivative) used in the present invention is a chimeric antibody or a humanized antibody derived from the mAb166 antibody (or a derivative thereof).

Methods for humanizing antibodies are well known to the skilled person.

The choice of the human variable domains of the heavy and light chains to be used to produce the humanized antibodies is important to reduce immunogenicity. According to the best-fit method, the sequence of the variable domain of the antibody of interest is compared with a library of known human variable domain sequences. The human sequence closest to the murine sequence is then selected as the human framework sequence (FR) for the humanized antibody (Sims et al, J. Immunol. 151, pp. 2296 (1993); Chothia and Lesk, J. Mol. Biol. 196, pp. 901).

Another method uses a particular FR sequence derived from a consensus sequence of all human antibodies of a particular heavy or light chain subgroup. The same FR sequence can be used for several different humanized antibodies (Carter et al., PNAS 89, pp. 4285 (1992); Presta et al. J. Immunol., 151 (1993)).

According to one embodiment, the at least one antibody (or antibody derivative) used in the present invention is an antibody (or antibody derivative) binding to the same epitope as mAb166. According to one embodiment, the at least one antibody (or antibody derivative) used in the present invention binds to a fragment of perV comprising amino acids 144 to 257 of perV (SEQ ID NO: 9).

According to the present invention, the infection is said to be respiratory if it affects at least one of the structures forming the respiratory system, i.e., the nose, throat, larynx, trachea, bronchi or lungs.

According to one embodiment, the respiratory infection is characterized by at least one of the following symptoms: dry cough, wet cough, moderate to high fever, chills, breathing disorders (e.g., shortness of breath), presence of a wheezing sound when breathing, chest pain, fatigue, nasal discharge, temporary loss of taste and smell.

According to one embodiment, the respiratory infection is caused by an infectious agent as described above. Thus, according to one embodiment, the respiratory infection is a Pseudomonas aeruginosa infection.

According to one embodiment, the respiratory infection is a lower respiratory tract infection, i.e., a respiratory infection affecting the lower airways or lungs.

According to one embodiment, the respiratory infection is an acute respiratory infection.

Non-limiting examples of lower respiratory tract infections include bronchitis, bronchiolitis, pneumonia (e.g., nosocomial pneumopathy, community-acquired pneumopathy, or ventilator-assisted pneumopathy), influenza, and pertussis.

Thus, according to one embodiment, the respiratory infection is a nosocomial pneumonia (or pneumopathy).

According to one embodiment, the respiratory infection is a primary infection, i.e., the subject has not been previously treated with a combination of the present invention for an infection with the same infectious agent or with an infectious agent of the same species.

According to one embodiment, the respiratory infection is a reinfection (or secondary infection), for example the second reinfection, or a repeated reinfection, i.e., a new infection caused by the same infectious agent or an infectious agent of the same species as defined above.

According to one embodiment, the respiratory infection is not a primary infection within the meaning of the present invention, i.e., the subject has been previously treated with a combination of the present invention for an infection with the same infectious agent or with an infectious agent of the same species.

According to one embodiment, the combination of the present invention treats a respiratory infection in a subject.

According to one embodiment, the combination of the present invention prevents the occurrence or recurrence of a respiratory infection in a subject.

According to one embodiment, the combination of the present invention prevents the occurrence of a respiratory infection in a subject in a short term. According to the present invention, prevention is said to be short-term when the at least one agent capable of binding the infectious agent, preferably an antibody or antibody derivative, is still detected in the body after administration of the combination.

According to one embodiment, the combination of the present invention induces a vaccinal effect (i.e., a long-term protection or prevention) preventing the occurrence or recurrence of a respiratory infection in a subject. According to the present invention, the protection or prevention is said to be long-term when the protection persists while the at least one agent capable of binding the infectious agent, preferably an antibody or antibody derivative, present in the combination of the invention is no longer detected in the organism and/or when an adaptive immune response is induced in the organism.

According to one embodiment, the long-term protection or prevention lasts at least 60, at least 70, at least 80, at least 90, or at least 100 days after administration of the combination, preferably at least 100 days.

In the sense of the present invention, the vaccinal effect is characterized by the ability of a combination as described above to induce an adaptive immune response allowing its action to be prolonged beyond the disappearance of the at least one agent capable of binding the infectious agent, preferably an antibody or an antibody derivative, present in the combination from the organism by stimulating specific immune responses.

Thus, according to one embodiment, the combination of the present invention induces an adaptive immune response to prevent the occurrence or recurrence of a respiratory infection in a subject.

Thus, one of the particularities of the present invention is that the administration of the combination of the present invention prevents and/or treats primary infections (short-term effect) but also reinfections (or secondary infections), possibly repeated (long-term effect) when the at least one agent capable of binding the infectious agent, preferably an antibody or an antibody derivative, present in the combination of the invention is no longer detected in the body.

According to one embodiment, the subject is considered treated if the number of infectious agents in said subject is decreased after administration of the combination of the present invention. According to one embodiment, the subject is considered treated if at least one of the symptoms of the respiratory infection decreases or disappears completely. According to one embodiment, the subject is considered treated if the morbidity associated with the respiratory infection is reduced or absent.

According to one embodiment, the subject is considered treated if, after receiving a therapeutically effective amount of the combination of the present invention, said subject develops a less severe respiratory infection than in the absence of treatment upon contact with the infectious agent targeted by said combination.

According to one embodiment, the subject is considered treated if, after receiving a therapeutically effective amount of the combination of the present invention, said subject does not develop a respiratory infection upon contact with the infectious agent targeted by said combination.

According to one embodiment, the subject is a mammal, preferably a human.

According to one embodiment, the subject is a male. According to another embodiment, the subject is a female.

According to one embodiment, the subject is an adult (>18 years old). According to another embodiment, the subject is a child (<18 years old).

According to one embodiment, the subject has a respiratory infection.

According to one embodiment, the subject is at risk of developing a respiratory infection.

Risk factors for developing a respiratory infection include, but are not limited to, age (children and persons over 65 years of age), smoking, alcohol abuse, drug use, living in substandard housing, pollution, having a chronic respiratory condition, airborne contamination or contact with a sick subject, and a nosocomial infection (including, for example, ventilator-assisted pneumopathy and nosocomial pneumopathy).

According to one embodiment, the subject suffers (preferably is diagnosed as suffering) from a chronic respiratory pathology.

According to one embodiment, said chronic respiratory pathology is selected from the group comprising or consisting of chronic obstructive pulmonary disease (COPD), pulmonary interstitial diseases, lung cancer, asthma (adult and pediatric), bronchiectasis, rare and orphan lung diseases such as cystic fibrosis, and pulmonary vascular diseases.

According to one embodiment, said chronic respiratory pathology is selected from the group comprising or consisting of chronic obstructive pulmonary disease (COPD), bronchiectasis, rare and orphan lung diseases such as cystic fibrosis.

Non-limiting examples of interstitial lung diseases (also known as diffuse parenchymal lung diseases) include sarcoidosis, idiopathic pulmonary fibrosis (IPF), extrinsic allergic alveolitis (hypersensitivity pneumopathy), interstitial lung disease associated with connective tissue disease, pneumoconiosis, and interstitial lung disease due to certain drugs.

Non-limiting examples of rare and orphan lung diseases include cystic fibrosis, lymphangioleiomyomatosis (LAM), scleroderma, pulmonary alveolar proteinosis (PAP), hypereosinophilic lung disease, combined pulmonary emphysema and fibrosis syndrome, multiple cystic pulmonary disease (MCPD), primary pulmonary ciliary dyskinesia, organized lung disease, pulmonary vasculitis, non-smoking bronchiolitis and COPD, alveolar hemorrhagic syndromes, primary pulmonary lymphoproliferative syndromes, tracheopathies, hereditary hemorrhagic telangiectasia with pulmonary arteriovenous malformation (Rendu-Osler disease) and bronchopulmonary amyloidosis.

Non-limiting examples of pulmonary vascular disease include pulmonary hypertension.

According to one embodiment, the subject has cystic fibrosis.

According to one embodiment, the subject has chronic obstructive pulmonary disease (COPD).

The present invention also relates to a composition, pharmaceutical composition or medicament for use in the treatment or prevention of a respiratory infection in a subject, said composition, pharmaceutical composition or medicament comprising or consisting essentially of a combination as previously described.

As used in this invention, the term “consists essentially of”, in reference to a composition, pharmaceutical composition or medicament, means that the object of the invention is the sole therapeutic agent or agent having biological activity in said composition, pharmaceutical composition or medicament.

According to one embodiment, the pharmaceutical composition for use in the present invention further comprises at least one pharmaceutically acceptable excipient.

Non-limiting examples of pharmaceutically acceptable liquid excipients include distilled water, saline solution, aqueous glucose solution, alcohol for example ethanol, propylene glycol, and polyethylene glycol; and oily vehicles such as vegetable and animal oils, kerosene, or wax.

Non-limiting examples of pharmaceutically acceptable solid excipients include glucose, fructose, sucrose, maltose, yellow dextrin, white dextrin, maltodextrin, microcrystalline cellulose, calcium stearate, magnesium stearate, sorbitol, glucose syrup, lactose, citric acid, tartaric acid, malic acid, succinic acid, lactic acid, L-ascorbic acid alpha-tocopherol, glycerol, propylene glycol, sucroester, polyglycerol esters of fatty acids, sucroglycerides, mono, di and triglyceride behenates, carrageenan, gum arabic, casein, gelatin, pectin, agar, nicotinamide, amino acids, calcium salts and pigments.

The present invention also relates to the use of a combination as described above for the manufacture of a medicament for the treatment or prevention of a respiratory infection in a subject.

According to one embodiment, a dose of about 0.5 to 50 mg of the at least one agent capable of binding the infectious agent of the present invention, preferably an antibody or antibody derivative, per kilo of weight is to be administered (or is for administration) to the subject, preferably a dose of about 1 to 20 mg per kilo, more preferably a dose of about 2.5 to 5 mg per kilo.

According to one embodiment, a probiotic strain as described above is to be administered (or is for administration) to the subject in an amount between 10³ and 10⁹ cfu (colony forming unit).

According to one embodiment, each probiotic strain of a mixture of 2 probiotic strains as described above is to be administered (or is for administration) to the subject in an amount between 10³ and 10⁹ cfu. According to one embodiment, each probiotic strain of a mixture of 3 probiotic strains as described above is to be administered (or is for administration) to the subject in an amount between 10³ and 10⁹ cfu

According to one embodiment, a mixture of 2 probiotic strains as previously described is to be administered (or is for administration) at a ratio of 1:1, 1:2, 1:3, 1:4, 1:5, 1:6; 1:7, 1:8, 1:9 or 1:10.

According to one embodiment, chitosan as previously described is to be administered (or is for administration) at a dose of about 0.1 to about 50 mg/kg, preferably about 0.5 to about 20 mg per day per kilogram.

According to one embodiment, flagellin, a flagellin variant or a polypeptide comprising or consisting of one or more flagellin fragment(s) (i.e., “flagellin polypeptide”) as described above is to be administered (or is for administration) at a dose of about 1 μg to about 100 mg, preferably at a dose of about 1 μg to about 10 mg, more preferably at a dose of about 1 μg to about 1 mg. According to one embodiment, the dose of flagellin, a flagellin variant or a flagellin polypeptide is adapted to have locally a dose of 1 to 1000 μg of a flagellin, a flagellin variant or a flagellin polypeptide at the respiratory tract.

According to one embodiment, the at least one agent capable of binding the infectious agent, preferably an antibody or antibody derivative, and the at least one immunomodulatory agent are to be administered (or are for administration) simultaneously.

According to another embodiment, the at least one agent capable of binding the infectious agent, preferably an antibody or antibody derivative, and the at least one immunomodulatory agent are to be administered (or are for administration) separately in time.

According to one embodiment, the at least one immunomodulatory agent is to be administered (or is for administration) before the at least one agent capable of binding the infectious agent, preferably an antibody or antibody derivative.

According to one embodiment, a probiotic strain or a mixture of probiotic strains as described above is to be administered (or is for administration) before the at least one agent capable of binding the infectious agent, preferably an antibody or antibody derivative.

According to another embodiment, the at least one immunomodulatory agent is to be administered (or is for administration) after the at least one agent capable of binding the infectious agent, preferably an antibody or antibody derivative.

According to one embodiment, the at least one agent capable of binding the infectious agent, preferably an antibody or antibody derivative, is to be administered (or is for administration) in a single dose.

According to one embodiment, the at least one agent capable of binding the infectious agent, preferably an antibody or antibody derivative, is to be administered (or is for administration) in repeated doses, for example once, twice or three times.

According to one embodiment, the at least one immunomodulatory agent is to be administered (or is for administration) in a single dose.

According to one embodiment, the at least one immunomodulatory agent, preferably a probiotic strain or a mixture of probiotic strains, is to be administered (or is for administration) in repeated doses, for example once, twice or three times.

According to one embodiment, the at least one immunomodulatory agent, preferably a probiotic strain or a mixture of probiotic strains, is to be administered (or is for administration) for at least 1, 2, 3, 4, 5, 6, 7, 8, 9, 10, 11, 12, 13, 14 days (preferably consecutive days) or until the subject is completely cured.

According to one embodiment, the at least one agent capable of binding the infectious agent, preferably an antibody or antibody derivative, and the at least one immunomodulatory agent used in the present invention (preferably a probiotic strain or a mixture of probiotic strains), are to be administered (or are for administration) by identical routes of administration, i.e., by inhalation.

According to the present invention, the at least one agent capable of binding the infectious agent, preferably an antibody or antibody derivative, is in a form suitable for administration by inhalation. According to one embodiment, both the at least one agent capable of binding the infectious agent, preferably an antibody or antibody derivative, and the at least one immunomodulatory agent are in a form suitable for administration by inhalation.

Non-limiting examples of formulations suitable for inhalation include aerosols (liquids or solids suspended in a carrier gas) and sprays (liquids or solids) for nasal use.

According to another embodiment, the at least one agent capable of binding the infectious agent, preferably an antibody or antibody derivative, and the at least one immunomodulatory agent are to be administered (or are for administration) by different routes of administration.

According to one embodiment, the at least one immunomodulatory agent is to be administered (or is for administration) enterally, topically or parenterally (injectable route).

In the sense of the present invention, the enteral route includes the oral route (enteral per os), the buccal route, the sublingual route, the rectal route, the pulmonary route, the percutaneous route, the nasal route and the local routes.

As used in this invention, an “oral administration” is an administration into the oral cavity, followed by ingestion of a compound, which enters the systemic circulation following its intestinal absorption. Examples of formulations suitable for oral administration include, but are not limited to, tablets (including sustained release tablets), capsules, powders, granules, pills (including sugar-coated pills), capsules (including soft gelatin capsules), oral suspensions, drinking solutions, and similar forms.

As used in the present invention, a “buccal administration” is an administration into the oral cavity of a compound, which is not followed by ingestion of said compound, the absorption of which occurs through the oral tissues, such as, for example, the palate, sublingual tissue, or gums. Non-limiting examples of formulations suitable for buccal administration include a chewing gum, a patch, and a buccal spray.

As used in this invention, a “rectal administration” is an administration of a compound through the anus, which enters the systemic circulation through the rectal mucosa. Non-limiting examples of formulations suitable for rectal administration include suppositories, rectal capsules, enemas or ointments.

As used herein, “percutaneous administration”, or “transdermal administration” is the administration of a compound onto the skin, followed by absorption into the systemic bloodstream through adjacent skin tissue. Non-limiting examples of formulations suitable for transcutaneous administration include transdermal ointment, paste, ointment, gel, cream or patch.

As used in this invention, a “nasal administration” is the administration of a compound directly to the nasal mucosa. Non-limiting examples of formulations suitable for nasal administration include sprays, nasal drops, nasal ointment and nasal spray solutions.

As used in this invention, “topical administration” is the administration of a compound to a body surface, said compound not intended to pass into the circulatory system. Examples of formulations suitable for topical administration include, but are not limited to, compositions in liquid, paste, or solid form and, more particularly, in the form of aqueous solutions, eye drops, dispersions, sprays, or microcapsules, micro- or nanoparticles or polymeric or gel patches allowing controlled release.

Within the meaning of the present invention, the term “injectable route” includes in particular subcutaneous, intravenous (IV), intramuscular, intra-articular, intra-synovial, intracisternal, intrathecal, intrahepatic, intralesional or intracranial. Examples of formulations suitable for administration by injection include, but are not limited to, sterile aqueous solutions, dispersions, emulsions, suspensions, solid forms suitable for the preparation of solutions or suspensions by addition of a liquid prior to use such as, for example, powders.

According to one embodiment, the at least one immunomodulatory agent comprises one or more probiotic strains and is to be administered (or is for administration) by inhalation, orally (enteral per os) or nasally. Thus, according to this embodiment, the at least one immunomodulatory agent is in a form suitable for administration by inhalation, oral (enteral per os) or nasal route.

The present invention also relates to a kit of parts (which may also be referred to as a kit) comprising at least two parts, the first part comprising at least one agent capable of binding the infectious agent as described above and the second part comprising at least one immunomodulatory agent as described above, for use in treating or preventing a respiratory infection in a subject.

According to one embodiment, said kit of parts comprises at least one antibody, at least one antibody derivative or at least one antibody mimetic as described above and a probiotic strain as described above. According to one embodiment, said parts kit comprises at least one antibody or antibody derivative as described above and a mixture of probiotic strains as described above. According to one embodiment, said kit comprises at least one antibody directed to Pseudomonas aeruginosa and a mixture of the 2 probiotic strains deposited on Apr. 14, 2015 at the CNCM under the numbers CNCM I-4967 and CNCM I-4968.

According to one embodiment, said kit of parts comprises at least one antibody directed against Pseudomonas aeruginosa and a probiotic strain selected from the strains deposited on Apr. 14, 2015 at the Collection Nationale de Cultures de Microorganismes (CNCM) under the numbers CNCM I-4967 and CNCM I-4968 or on Apr. 16, 2018 under the number CNCM I-5314 or a mixture thereof.

According to one embodiment, the first part of the kit of parts comprises a pharmaceutical composition comprising or consisting essentially of at least one agent capable of binding the infectious agent as described above, preferably an antibody or antibody derivative, and at least one pharmaceutically acceptable excipient.

According to one embodiment, the second part of the kit of parts comprises a pharmaceutical composition comprising or consisting essentially of one or more probiotic strains as described above, one or more Toll-like receptor agonists, one or more NOD-like receptor agonists, one or more RIG-like receptor agonists, a cytokine or mixture of cytokines, a chemokine or mixture of chemokines, one or more adjuvants such as chitosan, flagellin, a CpG oligodeoxynucleotide (CpG ODN), α-galactosylceramide (α-Gal-Cer), aluminum salts (aluminum hydroxide, aluminum phosphate, and potassium aluminum sulfate) MF59, AS03, polyinosinic-polycytidylic acid, a polyphosphazene or an antibody directed against immune checkpoints such as CTLA-4, PD-1, PD-L1 or CD137 and at least one pharmaceutically acceptable excipient.

According to one embodiment, the second part of the kit of parts comprises a pharmaceutical composition comprising or consisting essentially of a flagellin, a flagellin variant or polypeptide comprising or consisting of one or more flagellin fragments (“flagellin polypeptide”) as described above and at least one pharmaceutically acceptable excipient.

Examples of pharmaceutically acceptable excipients are given above.

According to one embodiment, the first and second portions of said kit of parts are to be administered (or are for administration) simultaneously or in a temporally separated manner.

According to one embodiment, the second part (comprising at least one immunomodulatory agent as described above) of said parts kit is to be administered (or is for administration) before the first part (comprising at least one agent capable of binding the infectious agent as described above).

According to one embodiment, the second part (comprising at least one immunomodulatory agent as described above) of said parts kit is to be administered (or is for administration) after the first part (comprising at least one agent capable of binding the infectious agent as described above).

According to one embodiment, the first and second parts of said parts kit are to be administered (or are for administration) by identical administration routes. According to this embodiment, the first and second parts of said parts kit are to be administered (or are for administration) by inhalation.

According to one embodiment, the first and second parts of said parts kit are to be administered (or are for administration) by different administration routes. According to this embodiment, the first part of said kit (comprising at least one agent capable of binding the infectious agent as described above) is to be administered (or is for administration) by inhalation, and the second part (comprising at least one immunomodulatory agent as described above) is to be administered (or is for administration) by another route.

According to the present invention, the first part of said kit of parts is adapted for administration by inhalation. According to one embodiment, the first and second portions of said parts kit are adapted for administration by inhalation.

Non-limiting examples of formulations adapted for inhalation include aerosols (liquids or solids suspended in a carrier gas) and sprays (liquids or solids) for nasal administration.

According to one embodiment, the second part of said kit of parts (comprising at least one immunomodulatory agent as described above) is to be administered (or is for administration) enterally, topically or parenterally (injectable route).

According to one embodiment, the second part of said kit of parts is to be administered (or is for administration) orally, buccally, sublingually, rectally, pulmonary, percutaneously, nasally or by a local route.

According to one embodiment, the second part of said kit is to be administered (or is for administration) by subcutaneous, intravenous (IV), intramuscular, intra-articular, intra-synovial, intracisternal, intrathecal, intralesional or intracranial injection.

According to one embodiment, the second part of said kit of parts comprises one or more probiotic strains and is to be administered (or is for administration) by inhalation, orally (enteral per os) or nasally. Thus, according to this embodiment, the second part of said parts kit is in a form suitable for administration by inhalation, oral (enteral per os) or nasal route.

The present invention also relates to a method of treating or preventing a respiratory infection in a subject, wherein the method comprises administering at least one agent capable of binding the infectious agent and at least one immunomodulatory agent as previously described, the at least one agent capable of binding the infectious agent being administered (or being formulated for administration) by inhalation.

The present invention also relates to the use of at least one agent capable of binding the infectious agent and at least one immunomodulatory agent as previously described for the manufacture of a medicament for treating or preventing a respiratory infection in a subject, the at least one agent capable of binding the infectious agent being administered (or being formulated for administration) by inhalation.

According to one embodiment, the at least one immunomodulatory agent comprises or consists of one or more probiotic strains, preferably selected from Lactobacillacus rhamnosus, Lactobacillacus salivarius and/or genomically related species.

According to one embodiment, the at least one immunomodulatory agent is or comprises a mixture of a probiotic strain selected from the species Lactobacillacus rhamnosus and a probiotic strain selected from the species Lactobacillacus salivarius.

According to one embodiment, the at least one immunomodulatory agent comprises or consists of one or more probiotic strains, preferably selected from the species Lactobacillacus murinus and/or a genomically related species.

According to one embodiment, the at least one immunomodulatory agent is selected from the strains deposited at the CNCM on Apr. 14, 2015 under the numbers CNCM I-4967 and CNCM I-4968, and a mixture thereof. According to one embodiment, the at least one immunomodulatory agent is the strain deposited at the CNCM on Apr. 16, 2018 under CNCM number I-5314.

According to a particular embodiment, the immunomodulatory agent is or comprises a mixture of the 2 probiotic strains deposited at the CNCM on Apr. 14, 2015 under numbers CNCM I-4967 and CNCM I-4968. According to a particular embodiment, the at least one immunomodulatory agent is or comprises a mixture of the probiotic strain deposited at the CNCM on Apr. 14, 2015 under the number CNCM I-4967 and the probiotic strain deposited at the CNCM on Apr. 16, 2018 under the number CNCM I-5314. According to one embodiment, the at least one immunomodulatory agent is or comprises a mixture of the probiotic strain deposited with the CNCM on Apr. 14, 2015 under the number CNCM I-4968 and the probiotic strain deposited at the CNCM on Apr. 16, 2018 under CNCM number I-5314.

According to a particular embodiment, the immunomodulatory agent is or comprises a mixture of the 3 probiotic strains deposited at CNCM on Apr. 14, 2015 under numbers CNCM I-4967 and CNCM I-4968 and on Apr. 16, 2018 under CNCM number I-5314.

According to one embodiment, the infectious agent is a virus, preferably an influenza virus, RSV, SARS-CoV or SARS-CoV-2.

According to one embodiment, the infectious agent is a bacterium, preferably a bacterium resistant to one or more antibiotics (preferably from the ESKAPE group), preferably a bacterium selected from the group comprising Pseudomonas aeruginosa, Klebsiella pneumoniae, Staphylococcus aureus and Acinetobacter baumanii, more preferably Pseudomonas aeruginosa.

According to one embodiment, the at least one agent capable of binding the infectious agent is directed against a molecule present on the surface of the infectious agent, preferably a molecule expressed on the surface of Pseudomonas aeruginosa. According to one embodiment, the at least one agent capable of binding the infectious agent of the present invention is directed against a secretion system protein, preferably a type III secretion system protein such as, for example, perV.

According to one embodiment, the at least one agent capable of binding the infectious agent is a molecule selected from the group consisting of an antibody, an antibody derivative such as an antibody fragment, a multispecific antibody, a bispecific antibody, a single-domain antibody, a unibody or nanobody and an antibody mimetic.

According to one embodiment, the at least one agent capable of binding the infectious agent is a chimeric, humanized or human-derived antibody or a derivative thereof.

According to one embodiment, the at least one antibody or antibody derivative used in the method of the present invention is mAb166 antibody or a chimeric or humanized antibody derived from mAb166 antibody or a derivative thereof.

According to one embodiment, the respiratory infection is an acute respiratory infection, preferably a lower respiratory tract infection selected from bronchitis, bronchiolitis, pneumonia (e.g., nosocomial pneumopathy, community-acquired pneumopathy, or ventilator-assisted pneumopathy), influenza, and pertussis.

According to one embodiment, the subject suffers from a chronic respiratory pathology, preferably a chronic respiratory pathology selected from the group comprising chronic obstructive pulmonary disease (COPD), pulmonary interstitial diseases, lung cancer, asthma (adult and pediatric), bronchiectasis, rare and orphan lung diseases such as cystic fibrosis, and pulmonary vascular diseases.

According to one embodiment, the method of the present invention prevents the occurrence of a respiratory infection in a subject in the short term.

According to one embodiment, the method of the present invention induces a vaccinal effect (i.e., long-term protection or prevention) preventing the occurrence or recurrence of a respiratory infection in the subject.

According to one embodiment, the method of the present invention comprises administering a dose of about 0.5 to 50 mg of the at least one agent capable of binding the infectious agent, preferably an antibody or antibody derivative, per kilo of body weight, preferably a dose of about 1 to 20 mg per kilo, more preferably a dose of about 2.5 to 5 mg per kilo.

According to one embodiment, the method of the present invention comprises administering a probiotic strain as described above in an amount of between 10³ and 10⁹ cfu (colony forming unit).

According to one embodiment, the method of the present invention comprises administering a mixture of 2 probiotic strains as described above at a ratio of 1:1, 1:2, 1:3, 1:4, 1:5, 1:6; 1:7, 1:8, 1:9 or 1:10.

According to one embodiment, the method of the present invention comprises administering a mixture of probiotic strains as described above wherein each strain is administered at an amount between 10³ and 10⁹ cfu (colony forming unit).

According to one embodiment, the method of the present invention comprises administering the at least one agent capable of binding the infectious agent, preferably an antibody or antibody derivative, and the at least one immunomodulatory agent simultaneously.

According to another embodiment, the method of the present invention comprises administering the at least one agent capable of binding the infectious agent, preferably an antibody or antibody derivative, and the at least one immunomodulatory agent separately in time.

According to one embodiment, the method of the present invention comprises administering the at least one agent capable of binding the infectious agent, preferably an antibody or antibody derivative, in a single dose.

According to one embodiment, the method of the present invention comprises administering the at least one agent capable of binding the infectious agent, preferably an antibody or antibody derivative, in repeated doses, for example once, twice or three times.

According to one embodiment, the method of the present invention comprises administering a probiotic strain or a mixture of probiotic strains in a single dose.

According to one embodiment, the method of the present invention comprises administering a probiotic strain or mixture of probiotic strains in repeated doses, for example once, twice or three times.

According to one embodiment, the method of the present invention comprises administering a probiotic strain or a mixture of probiotic strains for at least 1, 2, 3, 4, 5, 6, 7, 8, 9, 10, 11, 12, 13 or 14 days (preferably consecutive days).

According to one embodiment, the at least one agent capable of binding the infectious agent, preferably an antibody or antibody derivative, and the at least one immunomodulatory agent, preferably a probiotic strain or a mixture of probiotic strains, are administered by identical routes of administration, i.e., by inhalation.

According to another embodiment, the at least one agent capable of binding the infectious agent, preferably an antibody or an antibody derivative, and the at least one immunomodulatory agent, preferably a probiotic strain or a mixture of probiotic strains, are administered by different routes of administration.

According to one embodiment, a probiotic strain or mixture of probiotic strains of the present invention are administered by inhalation, orally (enteral route per os) or nasally.

### BRIEF DESCRIPTION OF THE DRAWINGS

FIG. 1 is a combination of drawings showing the experimental protocol for prophylactic treatment with antibodies and/or antibodies and probiotics. FIG. 1A: C57BL6/jRj mice (male, 7 weeks old), receive 50 μg of mAb166 via an intratracheal administration (Microsprayer®). 2 hours later, they are infected with Pseudomonas aeruginosa strain PA103, at a dose of 5×10⁵ cfu (colony forming unit)/40 μL/mouse via intratracheal administration. Clinical follow-up (survival, weight loss, clinical signs) of the animals is performed for 1 month after this primary infection. At D+33, the surviving individuals are reinfected, without additional treatment. Clinical follow-up is performed over 7 days. FIG. 1B: A cocktail of CNCM-I-4967 and CNCM-I-4968 strains, at 1×10⁶ cfu for each strain, is administered to animals at D (day)-3, D-2, D-1 intranasally (40 μL/mouse), prior to antibody treatment and infection as described in A.

FIG. 2 is a graph showing the survival of individuals to primary infection and reinfection with PA103 after prophylactic treatment with mAb166. Survival of individuals treated, according to the protocol described in FIG. 1, with mAb166 at 50 μg or a probiotic cocktail, or mAb166 at 50 μg combined with a probiotic cocktail or untreated, was analyzed after primary infection and reinfection. Results are representative of a pool of 5 independent experiments (n=10-30). (mAb166-50 μg+probiotics or mAb166-50 μg vs untreated groups ***: p<0.001/mAb166-50 μg+probiotics vs mAb166-50 μg groups ##: p<0.01, with a log-rank test).

FIG. 3 is a graph showing the pharmacokinetic analysis of mAb166 after prophylactic administration. The dosage of total mAb166 in serum and bronchoalveolar lavage (representative of the lower airways) was performed by ELISA at 1, 3, 7, 14, 21, and 28 days after antibody administration and PA103 infection. Data are expressed as mean±SEM (standard error of the mean). Results are representative of a pool of 3-5 independent experiments (n=5-30/analysis point). The dashed line indicates the limit of detection (ldd) of the assay.

FIG. 4 is a combination of drawings showing the experimental protocol of therapeutic treatment with antibody and/or antibody and probiotics. FIG. 4A: C57BL6/jRj mice (male, 7 weeks old) are infected with Pseudomonas aeruginosa strain PA103 at a dose of 3×10⁵ufc/40 μL/mouse via intratracheal administration and 1 hour later receive 50 μg of mAb166 via intratracheal administration (Microsprayer®). A clinical follow-up (survival, weight loss, clinical signs) of the animals is carried out during 1 month after this primary infection. At D+33, the surviving animals are reinfected, without additional treatment. Clinical follow-up is performed over 7 days. FIG. 4B: CNCM-I-4967, CNCM-I-4968, or CNCM-I-5314 at 1×1⁰⁵ cfu for each strain, are administered to animals at D-3, D-2, D-1 intranasally (40 μL/mouse), prior to infection and antibody treatment as described in A.

FIG. 5 is a combination of graphs showing survival of individuals to primary infection and reinfection with PA103 after therapeutic treatment with mAb166. FIG. 5A: CNCM-I-4967 strain: Results are representative of a pool of 3 independent experiments (n=5-50). (mAb166-50 μg+4967 or mAb166-50 μg vs untreated groups *: p<0.05; ***: p<0.001/mAb166-50 μg+4967 vs mAb166-50 μg groups ###: p<0.001, with a log-rank test). FIG. 5B: CNCM-I-4968 strain: Results are representative of a pool of 3 independent experiments (n=5-30). (mAb166-50 μg+4967 or mAb166-50 μg vs untreated groups *: p<0.05; ***: p<0.001/mAb166-50 μg+4967 vs mAb166-50 μg groups ###: p<0.001, with a log-rank test). FIG. 5C: CNCM-I-5314 strain: Results are representative of a pool of 3 independent experiments (n=5-35). (mAb166-50 μg+4967 or mAb166-50 μg vs untreated groups *: p<0.05; ***: p<0.001/mAb166-50 μg+4967 vs mAb166-50 μg groups ###: p<0.001, with a log-rank test).

FIG. 6 is a graph showing the pharmacokinetic analysis of mAb166 after therapeutic administration. The dosage of total mAb166 in serum and bronchoalveolar lavage (representative of the lower airways) was performed by ELISA at 1, 3, 7, 14, 21, and 28 days after antibody administration and infection with PA103. Data are expressed as mean±SEM. Results are representative of a pool of 3-5 independent experiments (n=5-30/analysis point). The dotted line indicates the limit of detection (ldd) of the assay.

FIG. 7 is a graph showing the anti-PA103 humoral response in serum to reinfection with PA103 after therapeutic treatment with mAb166. The amount of PA103-specific immunoglobulin G in individuals treated, according to the protocol described in FIG. 4B, with mAb166 at 50 μg, or mAb166 at 50 μg combined with a probiotic strain or mAb166 at 50 μg combined with Flagellin (FLAMOD, 2.5 μg/mouse), was analyzed after reinfection by a semi-quantitative ELISA. Results are representative of a pool of 2 independent experiments (n=5-10). (mab166 vs combo groups *: p<0.05; **: p<0.01, with an Anova test). The dotted line indicates the limit of detection (ldd) of the assay.

### EXAMPLES

The present invention will be better understood by reading the following example which illustrates the invention non-limitatively.

### Example 1: Prophylactic treatment with antibodies and/or antibodies and probiotics

### Materials and Methods

### Experimental Protocol

In these experiments, the Pseudomonas aeruginosa strain PA103, was used as representative of strains involved in acute respiratory infections.

C57/BL6jRj mice (male, 7 weeks old) were infected once with PA103 with a pulmonary deposition of the bacterial inoculum (primary infection). Bacteria were diluted in PBS to a titer of 5.10⁵ bacteria/40 μL.

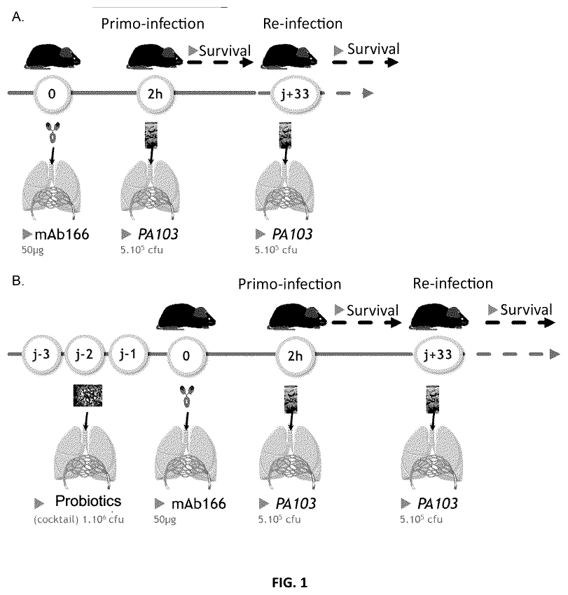

Two hours before, mice were administered by inhalation the mAb166 antibody (50 μg), a control IgG2b (MPC11 clone), or PBS solution via intratracheal administration using a MicroSprayer® aerosolizer (Penn-Century, USA) (FIG. 1A). mAb166 is a murine IgG2b specific for the perV protein, an essential component of the type 3 secretion system expressed on the surface of PA103.

For treatment of mice with probiotic strains, CNCM I-4967 and CNCM I-4968, derived from naïve mouse lungs and deposited on Apr. 14, 2015 at CNCM, were used. These strains were initially identified as belonging to Lactobacillus rhamnosus and Lactobacillus salivarius species, respectively, or genomically related species. Sequence analyses showed that these strains belong to the species Lactobacillus murinus.

Mice received a mixture of the probiotic strains CNCM I-4967 and 4968 (10⁶ cfu of each strain, 40 μL per mouse) by inhalation by intranasal route1, 2, and 3 days before priming (FIG. 1B).

After infection, survival and weight changes were monitored daily. In order to interrogate long-term protection induced by mAb166 antibody, animals surviving the primary infection were reinfected (secondary infection) at D+33 after the primary infection, without additional treatment. Their survival and the associated immune response are analyzed.

### Pharmacokinetic Analysis of mAb166 after Prophylactic Administration

The determination of total mAb166 in serum and bronchoalveolar lavage (representative of the lower airways) was performed by ELISA at 1, 3, 7, 14, 21, and 28 days after antibody administration and infection with PA103.

### Results

### Short-Term Protection Induced by Inhaled Antibody against Pa (Pseudomonas aeruginosa) in Combination with Probiotic Strains

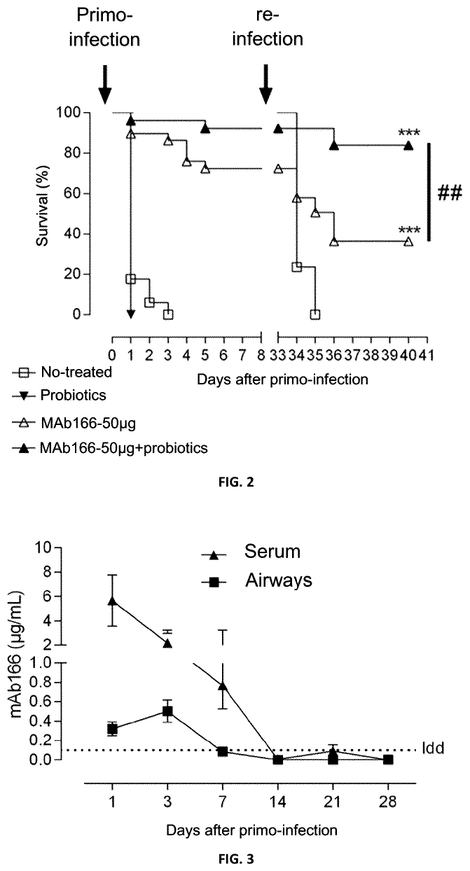

As shown in FIG. 2, the data reveal that animals treated with the anti-Pa mAb166 (50 μg) antibody administered by inhalation show a very significant improvement in survival compared to untreated animals (i.e., receiving the PBS solution) during primary infection. However, this protection is only partial, as the survival rate is about 70%. In addition, data show that administration of a control IgG2b (clone MPC11) has no positive impact on survival of treated animals during primary infection (data not shown).

These data also reveal that animals treated with a combination of the inhaled anti-Pa mAb166 antibody (50 μg) with a mixture of the probiotic strains CNCM I-4967 and CNCM-4968 show superior survival (greater than 90%) compared to untreated animals or animals treated with the anti-Pa mAb166 antibody alone during a first infection (FIG. 2). In contrast, administration of the probiotic strains CNCM I-4967 and CNCM-4968 alone does not affect survival.

Thus, the data indicate that the combination of an inhaled anti-Pa antibody with a mixture of probiotic strains is more effective in treating a first respiratory Pa infection.

### Long-Term Protection Induced by Inhaled Anti-Pa Antibody in Combination with Probiotic Strains

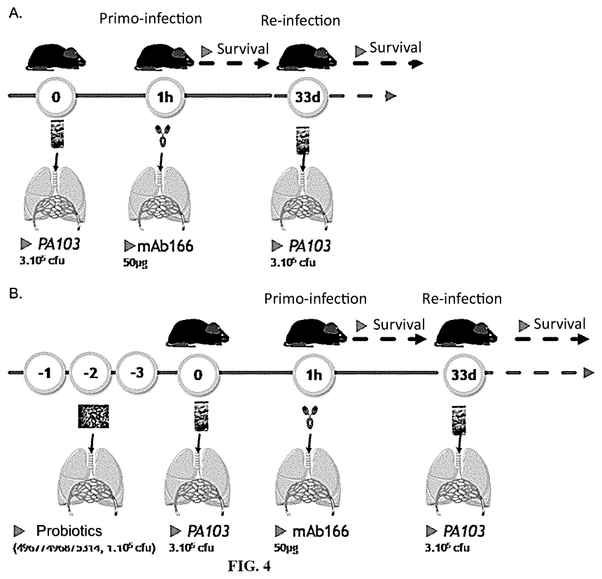

Surviving animals were reinfected at D+33 after primary infection, when anti-Pa mAb166 antibody was no longer detectable in blood and airways (FIG. 3), with the same dose of Pa as during primary infection.

Animals treated with the anti-Pa mAb166 antibody alone showed a better survival (less than 40%) after reinfection (or second infection) compared to untreated animals (FIG. 2), showing that Pa-infected animals treated with an inhaled antibody show a memory response allowing them to control a reinfection (or second infection) (by the same pathogen) in the absence of additional treatment during reinfection.

Interestingly, animals treated with the combination of the anti-Pa mAb166 antibody and the mixture of probiotic strains showed a significantly higher survival (around 80%) than animals that were not treated or were treated with the anti-Pa mAb166 antibody alone (FIG. 2). Administration of the probiotic strains alone had no significant effect. These data indicate that the combination of an inhaled antibody with the mixture of probiotic strains is more effective than the antibody alone in controlling reinfection (or second infection) with the same pathogen.

Thus, these data demonstrate that administration of a combination of an inhaled anti-infective antibody with a mixture of probiotic strains at the time of primary infection improves the short-term and long-term efficacy of an anti-infective antibody.

### Example 2: Therapeutic Treatment with Antibodies and/or Antibodies and Probiotics

### Materials and Methods

### Experimental Protocol

C57BL6/jRj mice (male, 7 weeks old) were infected with Pseudomonas aeruginosa strain PA103 at a dose of 3×10⁵ cfu/40 μL/mouse by inhalation via intratracheal administration and then received 1 hour later 50 μg of mAb166 via intratracheal administration (Microsprayer®) (FIG. 4A). Clinical follow-up (survival, weight loss, clinical signs) of the animals was performed for 1 month after this primary infection. At D+33, surviving individuals were reinfected, without additional treatment. Clinical follow-up was performed over 7 days.

For treatment of mice with probiotic strains, CNCM-I-4967, CNCM-I-4968, or CNCM-I-5314 at 1×10⁵ cfu for each strain, were administered to the animals at D-3, D-2, D-1 by inhalation by intranasal route (40 μL/mouse), prior to infection and antibody treatment as described in FIG. 4B.

### Pharmacokinetic Analysis of mAb166 after Therapeutic Administration

The dosage of total mAb166 in serum and bronchoalveolar lavage (representative of the lower airway) was performed by ELISA at 1, 3, 7, 14, 21, and 28 days after PA103 infection and antibody administration.

### Results

### Short-Term Treatment Induced by Inhaled Antibody to Pa (Pseudomonas aeruginosa) in Combination with Probiotic Strains

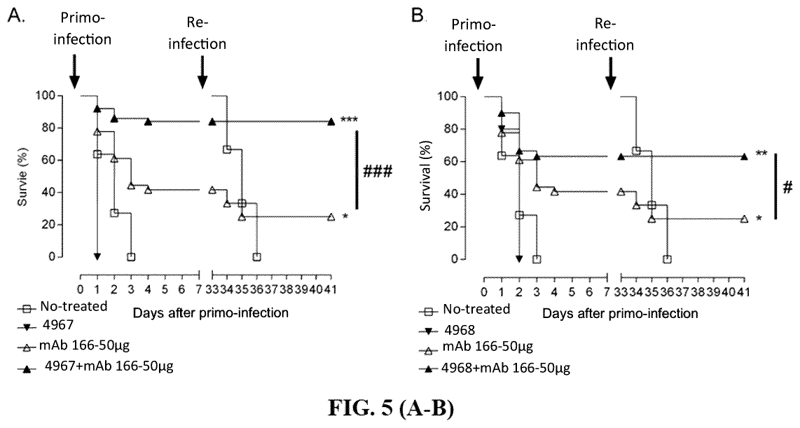

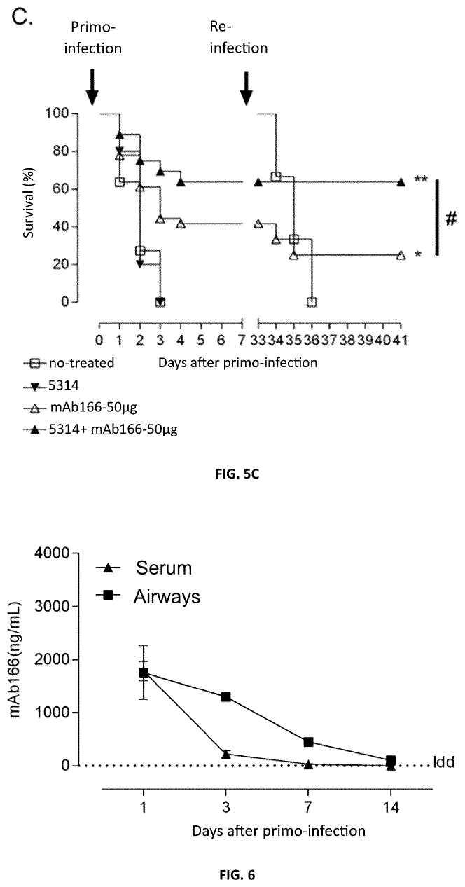

The results in FIG. 5A-C show that animals treated with the antibody in combination with one of the strains CNCM I-4967, CNCM I-4968, or CNCM I-5314 had better survival compared with untreated animals or animals treated with the mAb166 antibody alone during a first infection. In contrast, administration of one of the probiotic strains alone did not positively affect survival.

These data show that the combination of an inhaled anti-Pa antibody with a probiotic strain is more effective in treating a first Pa infection.

### Long-Term Treatment Induced by Inhaled Anti-Pa Antibody in Combination with Probiotic Strains

Surviving animals were reinfected at D+33 after primary infection, when mAb166 antibody was no longer detectable in blood and airways (FIG. 6), with the same dose of Pa as in the primary infection.

Animals treated with mAb166 antibody alone showed improved survival (about 30%) after reinfection (or second infection) compared to untreated animals (FIG. 5A-C), showing that Pa-infected animals treated with inhaled antibody have a memory response that allows them to control a second infection (with the same pathogen).

In contrast, animals treated with the combination of the mAb166 antibody and one of the CNCM I-4967, CNCM I-4968 or CNCM I-5314 strains showed a higher survival (between 60 and 80%) than animals that were not treated or were treated with the mAb166 antibody alone (FIG. 5A-C).

Thus, these data indicate that administration of a combination of an inhaled anti-Pa antibody with a probiotic strain at the time of primary infection can control both a first respiratory infection and also repeated respiratory infections when there are no inhaled anti-Pa antibodies or probiotic strains left in the body.

### Example 3: Humoral Response Induced during Reinfection with P. aeruginosa Following Treatment During Primary Infection with Anti-Pa Antibody, Anti-Pa Antibody Combined with a Probiotic Strain and Anti-Pa Antibody Combined with Flagellin

### Materials and Methods

The amount of PA103-specific immunoglobulin G in individuals treated, according to the protocol described in FIG. 4B, with mAb166 at 50 μg, or mAb166 at 50 μg combined with a probiotic strain or mAb166 at 50 μg combined with Flagellin (FLAMOD, 2.5 μg/mouse), was analyzed 5 days after reinfection by a semi-quantitative ELISA.

### Results

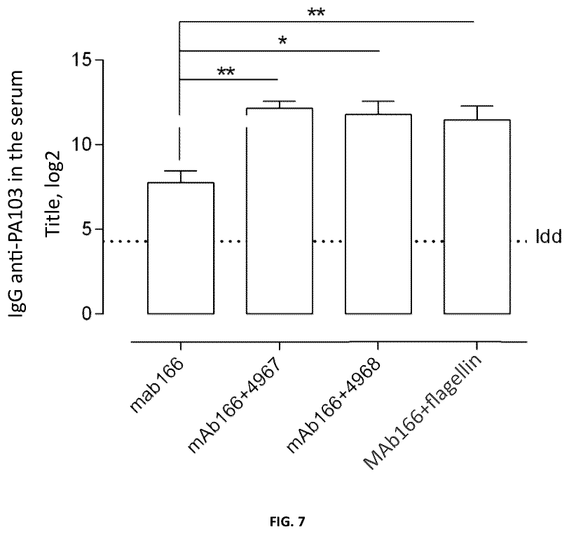

The results in FIG. 7 indicate that upon reinfection, the combination of an inhaled anti-Pa mAb166 antibody with a probiotic strain such as CNCM I-4967 and CNCM I-4968 induces a greater amount of PA103-specific immunoglobulin G in the serum of treated animals than treatment with an anti-Pa mAb166 antibody alone.

Interestingly, upon reinfection, the combination of inhaled anti-Pa mAb166 antibody in combination with flagellin also induces more PA103-specific immunoglobulin G in the serum of treated animals than treatment with anti-Pa mAb166 antibody alone (FIG. 7). Of note, individuals treated with flagellin alone during primary infection were not resistant to infection.

These results demonstrate that the humoral response upon reinfection is better when an inhaled anti-infective antibody was used in combination with a probiotic strain or flagellin than treatment with an antibody alone at the time of primary infection, thus effectively preventing recurrent respiratory infections in the long term

1-15. (canceled)

16. A method of treating or preventing a respiratory infection in a subject, wherein the method comprises administering at least one agent capable of binding an infectious agent and at least one immunomodulatory agent to the subject, wherein the at least one agent capable of binding the infectious agent is administered by inhalation.

17. The method according to claim 16, wherein the at least one agent capable of binding the infectious agent is selected from the group consisting of an antibody, an antibody derivative, and an antibody mimetic.

18. The method according to claim 16, wherein the at least one immunomodulatory agent is selected from the group consisting of a probiotic strain, a mixture of probiotic strains, a Toll-like receptor agonist, a NOD-like receptor agonist, a RIG-like receptor agonist, a cytokine or mixture of cytokines, a chemokine or mixture of chemokines, an adjuvant, a flagellin, a flagellin variant, a polypeptide comprising one or more flagellin fragment(s), a CpG oligodeoxynucleotide (CpG ODN), α-galactosylceramide (α-Gal-Cer), aluminum salts, MF59, AS03, polyinosinic-polycytidylic acid, a polyphosphazene, an antibody directed against immune checkpoints, and mixtures thereof.

19. The method according to claim 16, wherein the at least one immunomodulatory agent is or comprises a probiotic strain or a mixture of probiotic strains selected from the Lactobacillaceae family

20. The method according to claim 19, wherein the at least one immunomodulatory agent is or comprises a probiotic strain or a mixture of probiotic strains selected from the species Lactobacillus murinus.

21. The method according to claim 20, wherein the at least one immunomodulatory agent is or comprises a probiotic strain selected from the strains deposited at the Collection Nationale de Cultures de Microorganismes (CNCM) on Apr. 14, 2015 under numbers CNCM I-4967 and CNCM I-4968, or the strain deposited on Apr. 16, 2018 under number CNCM I-5314, or a mixture thereof.

22. The method according to claim 16, wherein the infectious agent is selected from the group comprising or consisting of viruses, bacteria, fungi and parasites.

23. The method according to claim 22, wherein the infectious agent is a bacterium.

24. The method according to claim 23, wherein the infectious agent is a bacterium from the group ESKAPE.

25. The method according to claim 16, wherein the at least one agent capable of binding the infectious agent is directed against a molecule present on the surface of a bacterium.

26. The method according to claim 25, wherein the at least one agent capable of binding the infectious agent is directed against a molecule present on the surface of Pseudomonas aeruginosa.

27. The method according to claim 16, wherein said respiratory infection is an acute respiratory infection.

28. The method according to claim 27, wherein said respiratory infection is an acute lower respiratory tract infection.

29. The method according to claim 28, wherein said respiratory infection is selected from bronchitis, bronchiolitis, pneumonia, nosocomial pneumopathy, community-acquired pneumopathy, ventilator-assisted pneumopathy, influenza, or pertussis.

30. The method according to claim 16, wherein the subject suffers from a chronic respiratory pathology.

31. The method according to claim 30, wherein the chronic respiratory pathology is selected from the group consisting of chronic obstructive pulmonary disease (COPD), pulmonary interstitial diseases, lung cancer, adult asthma and pediatric asthma, bronchiectasis, rare and orphan lung diseases, cystic fibrosis and pulmonary vascular diseases.

32. The method according to claim 16, wherein the at least one agent capable of binding the infectious agent and the at least one immunomodulatory agent are administered separately in time.

33. The method according to claim 16, wherein the at least one agent capable of binding the infectious agent and the at least one immunomodulatory agent are administered simultaneously.

34. A method of treating or preventing a respiratory infection in a subject, wherein the method comprises administering a composition comprising or consisting essentially of a combination of at least one agent capable of binding an infectious agent and at least one immunomodulatory agent, wherein the at least one agent capable of binding the infectious agent and the at least one immunomodulatory agent are administered by inhalation.

35. The method according to claim 34, wherein the composition is a pharmaceutical composition and further comprises at least one pharmaceutically acceptable excipient.
